(19) United States
(12) Patent Application Publication
(10) Pub. No.: US 2023/0139944 A1
(43) Pub. Date: May 4, 2023

(54) TARGETING ALPHA3BETA1 INTEGRIN FOR TREATMENT OF CANCER AND OTHER DISEASES

(71) Applicant: Board of Regents, The University of Texas System, Austin, TX (US)

(72) Inventor: Raghu KALLURI, Houston, TX (US)

(73) Assignee: Board of Regents, The University of Texas System, Austin, TX (US)

(21) Appl. No.: 17/621,134

(22) PCT Filed: Jun. 17, 2020
(86) PCT No.: PCT/US2020/038158

§ 371 Date: Dec. 20, 2021

Related U.S. Application Data
(60) Provisional application No. 62/864,611, filed on Jun. 21, 2019.

(51) Int. Cl. C07K 16/28; A61K 47/68; A61P 35/00; A61K 45/06; A61K 39/395; A61K 31/5377; C07K 14/705; C07K 14/725; A61K 35/17; A61K 31/713

(52) U.S. Cl. CPC C07K 16/2842; C07K 16/2809; A61K 47/6849; A61P 35/00; A61K 45/06; A61K 39/3955; A61K 39/39558; A61K 31/5377; C07K 14/70517; C07K 14/70521; C07K 14/7051; A61K 35/17; A61K 31/713; A61K 2039/505

(57) ABSTRACT

Provided herein, in some aspects, are agents, such as antibodies, chimeric antigen receptors, or RNA interference molecules that target the interaction between α3β1 integrin and α1 homotrimeric type I collagen. Aspects are directed to methods of treating cancer and fibroids comprising administering to a patient in need thereof an effective amount of an agent that disrupts the interaction between α3β1 integrin and α1 homotrimeric type I collagen. The methods can further include administering an effective amount of chemotherapy or immunotherapy to said patient.

This application claims the benefit of U.S. Provisional Patent Application No. 62/864,611, filed Jun. 21, 2019, which is expressly incorporated by reference herein in its entirety.

### BACKGROUND

### 1. Field

Aspects of the present invention relates generally to the field of medicine. Certain aspects concern methods of treating cancer by disrupting the interaction between homotrimeric type I collagen and α3β1 integrin.

### 2. Background

Type I collagen (col1), a fibrillar collagen, is the most abundant protein in the human body and most abundantly present in bones, tendons and skin. The basic functional unit of col1 is a heterotrimer consisting of two a1 chains and one a2 chain that come together to form a triple helical structure. Each a-chain polypeptide is synthesized in the cytosol and combines with two other a-chains to generate a triple-helical type I procollagen with N-terminal and C-terminal propeptides. Subsequently, the procollagen molecule is secreted into the extracellular space where the N-terminal and C-terminal pro-peptides are cleaved by propeptidases, generating the basic functional unit of Col1. The Col1 triple helical rod-like molecules interacts with each other to form fibrils and undergo further crosslinking to form large bundles of fibers.

During embryogenesis, many organs express col1 to likely facilitate cellular migration, differentiation and structural compartmentalization, but it is largely absent in adult tissue parenchyma and organs (Hay, 1981). Systemic deletion of Col1a1 gene (resulting in complete absence of type I collagen) leads to embryonic lethality (Lohler et al., 1984). In pathogenic conditions, such as organ fibrosis and cancer, Col1 accumulates robustly in the affected tissue (Apte et al., 2012; Armstrong et al., 2004; Bachem et al., 2005; Fujita et al., 2009; Haber et al., 1999). Col1 associated with tumor tissue is known to generate a biophysically ‘stiff’ environment around the cancer cells facilitating cellular migration via conducive fiber ‘tracks’, and facilitate abnormal cellular interactions to induce proliferation and survival of cancer cells (Apte et al., 2012; Armstrong et al., 2004; Bachem et al., 2005; Egeblad et al., 2010; Fujita et al., 2009; Haber et al., 1999; Levental et al., 2009). In this regard, Col1 is a major component of the tumor stroma/microenvironment associated with pancreatic cancer (Mollenhauer et al., 1987). aSMA⁺ myofibroblasts (MFs) associated with PDAC are speculated to significantly contribute to the production of Col1 and proposed to impede drug delivery to cancer cells (Apte et al., 2012; Armstrong et al., 2004; Bachem et al., 2005; Egeblad et al., 2010; Fujita et al., 2009; Haber et al., 1999; Levental et al., 2009; Provenzano et al., 2012). Recent studies suggest that stromal fibroblasts in PDAC might exhibit context dependent functions, imparting tumor promoting and restraining influences (Biffi et al., 2019; Kalluri, 2016; Laklai et al., 2016; Lee et al., 2014; Mueller and Fusenig, 2004; Neesse et al., 2015; Ohlund et al., 2014; Ohlund et al., 2017; Olive et al., 2009; Ozdemir et al., 2014; Provenzano et al., 2012; Rhim et al., 2014; Sugimoto et al., 2006). In this regard, precise function of activated stellate cells/myofibroblast produced type I collagen in initiation and progression of PDAC remains unknown.

### SUMMARY

In some embodiments, provided herein are compositions comprising an antibody or an antibody fragment that binds to α3β1 integrin. In some aspects, the antibody or antibody fragment binds to α3β1 integrin on epithelial cells. In some aspects, the antibody or antibody fragment binds to α3β1 integrin on fibroblasts. In some aspects, the antibody or antibody fragment disrupts the interaction between α3β1 integrin and α1 homotrimeric type I collagen. In some aspects, the antibody or antibody fragment inhibits pro-survival signaling through α3β1 integrin.

In some aspects, the antibody fragment is a recombinant scFv (single chain fragment variable) antibody, Fab fragment, F(ab′)₂ fragment, or Fv fragment. In some aspects, the antibody is a chimeric antibody or is a bispecific antibody. In some aspects, the chimeric antibody is a humanized antibody. In some aspects, the bispecific antibody binds to both α3β1 integrin and CD3. In some aspects, the antibody or antibody fragment is conjugated to a cytotoxic agent. In some aspects, the antibody or antibody fragment is conjugated to a diagnostic agent.

In some embodiments, provided herein are hybridomas or engineered cells encoding an antibody or antibody fragment of any one of the present embodiments. In one embodiment, provided herein are pharmaceutical formulations comprising one or more antibody or antibody fragment of any one of the present embodiments.

In some embodiments, provided herein are methods of treating a patient in need thereof, the methods comprising administering an effective amount of an α3β1 integrin-specific antibody or antibody fragment. In some aspects, the antibody or antibody fragment binds to α3β1 integrin on epithelial cells. In some aspects, the antibody or antibody fragment binds to α3β1 integrin on fibroblasts. In some aspects, the antibody or antibody fragment disrupts the interaction between α3β1 integrin and a1 homotrimeric type I collagen. In some aspects, the antibody or antibody fragment inhibits pro-survival signaling through α3β1 integrin. In some aspects, the α3β1 integrin-specific antibody or antibody fragment is the antibody or antibody fragment of any one of the present embodiments.

In some aspects, the patient has a cancer, a fibroid disease, a tissue injury, keloids, organ fibrosis, Crohn's disease, strictures, colitis, psoriasis, or a connective tissue disorder. In some aspects, the patient is need of tissue injury repair or tissue regeneration. In some aspects, the connective tissue disorder is a connective tissue disorder that involves collagen. In some aspects, the connective tissue disorder that involves collagen is a connective tissue disorder that involves type 1 collagen.

In certain aspects, the patient has a cancer. In some aspects, the cancer patient has been determined to express an elevated level of a1 homotrimeric type I collagen relative to a control patient. In some aspects, the cancer is a pancreatic cancer. In some aspects, the methods are further defined as methods of inhibiting pancreatic cancer metastasis. In some aspects, the methods are further defined as methods of inhibiting pancreatic cancer growth.

In some aspects, the methods further comprise administering at least a second anti-cancer therapy. In some aspects, the second anti-cancer therapy is a chemotherapy, immunotherapy, radiotherapy, gene therapy, surgery, hormonal therapy, anti-angiogenic therapy or cytokine therapy. In some aspects, the second anti-cancer therapy is an immunotherapy. In some aspects, the immunotherapy is a checkpoint blockade therapy. In some aspects, the checkpoint blockade therapy comprises administering an anti-PD-1 antibody or antibody fragment. In some aspects, the methods further comprise administering an inhibitor of integrin signaling. In some aspects, the inhibitor of integrin signaling inhibits FAK and/or PYK2. In some aspects, the inhibitor of integrin signaling is VS-4718 (PND-1086).

In some embodiments, provided herein are methods of treating a patient in need thereof, the methods comprising administering an effective amount of an agent that inhibits pro-survival signaling through α3β1 integrin. In some aspects, the agent is an antibody or antibody fragment disrupts the interaction between α3β1 integrin and a1 homotrimeric type I collagen. In some aspects, the α3β1 integrin-specific antibody or antibody fragment is the antibody or antibody fragment of any one of the present embodiments. In some aspects, the agent is an anti-sense oligonucleotide that inhibits the expression of α3β1 integrin.

In some aspects, the patient has a cancer, a fibroid disease, a tissue injury, keloids, organ fibrosis, Crohn's disease, strictures, colitis, psoriasis, or a connective tissue disorder. In some aspects, the patient is in need of tissue injury repair or tissue regeneration. In some aspects, the connective tissue disorder is a connective tissue disorder that involves collagen. In some aspects, the connective tissue disorder that involves collagen is a connective tissue disorder that involves type 1 collagen.

In some aspects, the patient has a cancer. In some aspects, the cancer patient has been determined to express an elevated level of a1 homotrimeric type I collagen relative to a control patient. In some aspects, the cancer is a pancreatic cancer. In some aspects, the methods are further defined as methods of inhibiting pancreatic cancer metastasis. In some aspects, the methods are further defined as methods of inhibiting pancreatic cancer growth.

In some aspects, the methods further comprise administering at least a second anti-cancer therapy. In some aspects, the second anti-cancer therapy is a chemotherapy, immunotherapy, radiotherapy, gene therapy, surgery, hormonal therapy, anti-angiogenic therapy or cytokine therapy. In some aspects, the methods further comprise administering an inhibitor of integrin signaling. In some aspects, the inhibitor of integrin signaling inhibits FAK and/or PYK2. In some aspects, the inhibitor of integrin signaling is VS-4718 (PND-1086).

In some embodiments, provided herein are methods of treating a subject comprising administering an anti-tumor effective amount of an agent that inhibits the expression of α3β1 integrin. In some aspects, the agent is an siRNA that targets α3β1 integrin mRNA. In some aspects, the agent is formulated in a lipid nanoparticle. In some aspects, the lipid nanoparticle is an exosome. In some aspects, the subject has a cancer. In some aspects, the cancer is a pancreatic cancer. In some aspects, the methods further comprise administering an inhibitor of integrin signaling. In some aspects, the inhibitor of integrin signaling inhibits FAK and/or PYK2. In some aspects, the inhibitor of integrin signaling is VS-4718 (PND-1086).

In some embodiments, provided herein are chimeric antigen receptor (CAR) polypeptides comprising, from N- to C-terminus, an antigen binding domain; a hinge domain; a transmembrane domain and an intracellular signaling domain, wherein the CAR polypeptide binds to an α3β1 integrin. In some aspects, the antigen binding domain comprises HCDR sequences from a first antibody that binds to an α3β1 integrin and LCDR sequences from a second antibody that binds to an α3β1 integrin. In some aspects, the antigen binding domain comprises HCDR sequences and LCDR sequence from an antibody that binds to an α3β1 integrin. In some aspects, the CAR disrupts the interaction between α3β1 integrin and a1 homotrimeric type I collagen. In some aspects, the hinge domain is a CD8a hinge domain or an IgG4 hinge domain. In some aspects, the transmembrane domain is a CD8a transmembrane domain or a CD28 transmembrane domain. In some aspects, the intracellular signaling domain comprises a CD3z intracellular signaling domain.

In some embodiments, provided herein are nucleic acid molecules encoding a CAR polypeptide of any one of the present embodiments. In some aspects, the sequence encoding the CAR polypeptide is operatively linked to expression control sequences.

In some embodiments, provided herein are isolated immune effector cells comprising a CAR polypeptide according to any one of the present embodiments or a nucleic acid of any one of the present embodiments. In some aspects, the nucleic acid is integrated into the genome of the cell. In some aspects, the cell is a T cell. In some aspects, the cell is an NK cell. In some aspects, the cell is a human cell.

In some embodiments, provided herein are pharmaceutical compositions comprising a population of cells in accordance with any one of the present embodiments in a pharmaceutically acceptable carrier.

In some embodiments, provided herein are methods of treating a subject comprising administering an anti-tumor effective amount of chimeric antigen receptor (CAR) T cells that expresses a CAR polypeptide in accordance with any one of the present embodiments. In some aspects, the CAR T cells are allogeneic cells. In some aspects, the CAR T cells are autologous cells. In some aspects, the CAR T cells are HLA matched to the subject. In some aspects, the subject has a cancer. In some aspects, the cancer is a pancreatic cancer. In some aspects, the methods further comprise administering an inhibitor of integrin signaling. In some aspects, the inhibitor of integrin signaling inhibits FAK and/or PYK2. In some aspects, the inhibitor of integrin signaling is VS-4718 (PND-1086).

In some embodiments, provided herein are methods of treating a subject comprising administering an anti-tumor effective amount of chimeric antigen receptor (CAR) NK cells that expresses a CAR polypeptide in accordance with any one of the present embodiments. In some aspects, the CAR NK cells are allogeneic cells. In some aspects, the CAR NK cells are autologous cells. In some aspects, the CAR NK cells are HLA matched to the subject. In some aspects, the subject has a cancer. In some aspects, the cancer is a pancreatic cancer. In some aspects, the methods further comprise administering an inhibitor of integrin signaling. In some aspects, the inhibitor of integrin signaling inhibits FAK and/or PYK2. In some aspects, the inhibitor of integrin signaling is VS-4718 (PND-1086). In some aspects, the methods further comprise administering at least a second anti-cancer therapy. In some aspects, the second anti-cancer therapy is a chemotherapy, immunotherapy, radiotherapy, gene therapy, surgery, hormonal therapy, anti-angiogenic therapy or cytokine therapy. In some aspects, the methods further comprise administering an inhibitor of integrin signaling. In some aspects, the inhibitor of integrin signaling inhibits FAK and/or PYK2. In some aspects, the inhibitor of integrin signaling is VS-4718 (PND-1086).

In some embodiments, provided herein is a method of treating a subject for cancer, the method comprising (a) administering to the subject an effective amount of an α3β1 integrin-specific antibody or antibody fragment; (b) administering to the subject an anti-tumor effective amount of chimeric antigen receptor (CAR) T cells that expresses a CAR polypeptide comprising, from N- to C-terminus, an antigen binding domain; a hinge domain; a transmembrane domain and an intracellular signaling domain, wherein the CAR polypeptide binds to an α3β1 integrin; or (c) administering to the subject an anti-tumor effective amount of an agent that inhibits the expression of α3β1 integrin; wherein cancer cells from the subject have been determined to have an increased expression of α3 integrin relative to healthy or control cells. In some embodiments, the method comprises administering to the subject the effective amount of the α3β1 integrin-specific antibody or antibody fragment of (a), wherein the antibody or antibody fragment disrupts the interaction between α3β1 integrin and a1 homotrimeric type I collagen. In some embodiments, the method comprises administering to the subject the anti-tumor effective amount of the chimeric antigen receptor (CAR) T cells of (b). In some embodiments, the method comprises administering to the subject the anti-tumor effective amount of the agent of (c).

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. Type I collagen homotrimers induce pro-survival signaling through α3β1 integrin. KPPC; Col1^{pdxKO} cancer cells were transfected with siRNAs of DDR1 (A,B), integrin β1 (C), or integrin α3 (D). Cells were then incubated with DMEM medium with 1% FBS for 6 hours before the treatment with col1 homotrimers or heterotrimers (50 μg/mL) for 16 hours. Cells were harvested, lysed, and examined for indicated proteins by Western blotting.

FIGS. 2A-2H. Type I collagen homotrimers induce persistent proliferation signals via integrins. (A,B) KPPC; Col1^{pdxKO} cancer cells were transfected with siRNAs of integrins β1 (A), α1, α2, or α3 (B). Cells were then incubated with DMEM medium with 1% FBS for 6 hours before the treatment with col1 homotrimers or heterotrimers (50 μg/mL) for 16 hours. Cells were harvested, lysed, and examined by Western blotting. (C) Expression levels of selected integrin subunits in KPPC; Col1^{pdxKO} cancer cells. (D) Expression levels of selected integrin subunits human PDAC cell lines (Panc1, BxPC3, MiaPaca2, PSN1, HPAC, CAPAN1) based on RNA-seq data from the Broad Institute Cancer Cell Line Encyclopedia (CCLE) database. RMA (Robust Multi-array Average algorithm) normalization was used and data are the log 2 gene expression signal. (E) Expression profile of integrin α3 (Itga3) among various cell clusters from KPPC tumors, as shown in t-SNE plot using the single cell RNA sequencing analysis. (F) Representative images of integrin α3 IHC staining on KPPC tumor sections. Scale bar: 100 μm. (G) KPPC; Col1^{pdxKO} cancer cells growing in 6-well plates were cultured with regular medium (DMEM with 10% FBS) to reach 60% confluence. Cells were then incubated with DMEM medium with 1% FBS for 6 hours before the treatment with col1 homotrimers or heterotrimers (50 μg/mL) in the presence or absence of FAK inhibitor (VS-4718, 1 μM) for 16 hours. Cells were harvested, lysed, and examined for cell signaling by Western blotting. (H) H&E-stained sections of pancreas of KPPC (age-matched 4-week-old) mice treated with vehicle or FAK inhibitor (VS-4718, oral gavage treatment, 50 mg/kg twice per day for 7 days). Scale bar: 100 m. Percent area of ADM and PanIN lesions was quantified. * P<0.05.

FIGS. 3A-3X. Single cell RNA sequencing (sc-RNA-seq) analysis of unfractionated live cell mixture from pancreatic tumors of KPPC (LSL-Kras^{G12D}; Trp53^{loxP/loxP}; Pdx1-Cre) mice, showing the expression patterns of integrin subunits in indicated cell clusters. Heat map of top upregulated gene signatures of functional cell clusters 1-13 is shown. Continued from FIG. 2E.

FIG. 4. Representative images of integrins a3 IHC staining on murine and human pancreatic tumor tissue sections. Continued from FIG. 2F. Scale bar: 100 μm.

FIGS. 5A-5E. Integrin α3 immunochemical staining on tissue microarray. FIG. 5A shows representative images showing the immunohistochemical (IHC) scores of integrin α3 on a scale of 0-3 in human PDAC sections. The staining intensity of integrin α3 was quantified by visual scoring of staining (3—very high, 2—high, 1—low, and 0—negative). FIG. 5B shows IHC scores of integrin α3 for all samples graded by combined score of the intensity of staining and the percentage of positive tumor cells. The average score of integrin α3 expression was 1.87 for the entire cohort. FIG. 5C shows a table of case number and percentage of PDAC samples with indicated integrin α3 expression levels (score 2-3: very high, score 1-2: high, and score <1: low). FIGS. 5D and 5E show Kaplan-Meier survival curves showing the correlation between integrin α3 expression level and overall survival (FIG. 5D) and progression-free survival (FIG. 5E). The expression of integrin α3 was categorized as ITGA3—high (lower line; n=68) and ITGA3-low (upper line; n=62) using the average combined score 1.87 as a cutoff.

FIG. 6. In vitro a3 integrin (Itga3) siRNA treatment. KPPC and KPPC; Col1^{pdxKO} cancer cells were transfected with siRNA of integrin α3. Cells were then incubated with RPMI medium with 1% FBS for 48 hours and examined by cell viability assay (n=3 biological replicates). ** P<0.01, NS: not significant.

FIG. 7. Exosome-mediated Itga3 siRNA delivery to KPCC mice. Survival of KPPC (5-week-old) mice treated with mesenchymal stem cell-derived exosomes electroporated with either siRNA-control or siRNA-Itga3 (intraperitoneal injection with 10⁸ exosomes per injection containing 0.1 μg of siRNAs every 48 hours). ** P<0.01.

FIGS. 8A-8H. Deletion of type I collagen homotrimers by cancer cells increases T cell infiltration and enables efficacy of anti-PD-1 therapy. FIGS. 8A and 8B show schematic representations of the genetic modifications of KPFF (FIG. 8A) and KPFF; Col1^{smaKO} (FIG. 8B) mice. FIG. 8C shows RNA-seq on KPPF tumors (n=3) and KPPF; Col1^{smaKO} tumors (n=4). Ingenuity pathway analysis (IPA) was performed to visualize downregulated immune pathways in KPPF; Col1^{smaKO} tumors, when compared to KPPF tumors. These genes are also listed with log 2-fold change and P value. FIGS. 8D and 8E show schematic representations of the genetic modifications of KPPC (FIG. 8D) and KPPC; Col1^{pdxKO} (FIG. 8E) mice. FIG. 8F shows RNA-seq on KPPC tumors (n=3) and KPPC; Col1^{smaKO} tumors (n=4). Ingenuity pathway analysis (IPA) was performed to visualize downregulated immune pathways in KPPC; Col1^{pdxKO} tumors, when compared to KPPC tumors. These genes are also listed with log 2-fold change and P value. FIG. 8G shows CD3⁺, CD4⁺, and CD8⁺ T cell quantification from multispectral imaging of multiplex stained sections of KPPF tumors (n=4) and KPPF; Col1^{smaKO} tumors (n=4). Scale bar: 100 μm. FIG. 8H shows CD3⁺, CD4⁺, and CD8⁺ T cell quantification from multispectral imaging of multiplex stained sections of KPPC tumors (n=8) and KPPC; Col1^{pdxKO} tumors (n=6). Scale bar: 100 μm. * P<0.05, ** P<0.01, *** P<0.001, NS: not significant.

FIG. 9A-9N. Deletion of type I collagen homotrimers by cancer cells reverses the immunosuppressive impact on T cell infiltration and enables efficacy of anti-PD-1 therapy. FIGS. 9A and 9B show flow cytometry analyses of percentage immune cell populations (positive for indicated markers) in KPPC (FIG. 9A) and KPPC; Col1^{pdxKO}(FIG. 9B) tumors (from 5 mice per group). Gating strategy for CD4⁺ and CD8⁺ population out of CD3⁺ cells is shown. FIGS. 9C-9H show quantification of % CD45+ (FIG. 9C), % CD3+ (FIG. 9D), % CD4+ (FIG. 9E), % PD1+CD4+ (FIG. 9F), % CD8+ (FIG. 9G), and % PD1+CD8+ (FIG. 9H) cells. FIG. 9I shows survival of KPPC mice (n=6) and KPPC; Col1^{pdxKO} mice (n=10) treated with anti-PD1 therapy, compared to KPPC (n=26) and KPPC; Col1^{pdxKO} (n=33) mice. FIG. 9J shows a schematic of experimental methods of the present disclosure. FIGS. 9K-9N show results from studies where splenic lymphocytes (isolated from 4 healthy mice) were cultured in the presence or absence of: activation (anti-CD3/anti-CD28 antibodies, 1 μg/mL), KPPC cancer cell co-culture, and KPPC; Col1^{pdxKO} cancer cell co-culture for 1 day (lymphocyte:cancer cell ratio=10:1). Splenic lymphocytes were then examined by flow cytometry. Bar graphs show results from the following conditions, from left to right for each bar graph: No activation, Activation, Activation and KPCC co-culture, and Activation and KPCC; Col1^{pdxKO} co-culture.

### DETAILED DESCRIPTION

Aspects of the present disclosure relate to the fact that cancer cells specifically produce a variant of type 1 collagen. Without wishing to be bound by theory, it is understood that healthy myofibroblasts produce an α1/α2/α1 heterotrimer, which binds to DDR receptors and restrains tumor growth. On the other hand, as disclosed herein, cancer cells produce an α1/α1/α1 homotrimer, which binds to α3β1 integrins thereby inducing pro-oncogenic signals.

Col1 homotrimers robustly induce phosphorylation of DDR1 and activate FAK, AKT and ERK1/2 when compared to heterotrimers. On first assessment, this stands in contrast to what has been published before (Armstrong et al., 2004; Bachem et al., 2005; Fujita et al., 2009). But in all of the such studies the contribution of Col1 produced by cancer cells cannot be ruled out. Suppression of DDR1 leads to continued activation of FAK, AKT, and ERK1/2. This suggests that Col1 homotrimers can activate other receptors in parallel or as a compensatory mechanism. Single cell RNA sequencing analysis suggested that pancreatic cancer cells can express Col1-binding integrins such as all, α2β1, and α3β1. Previous studies have shown that integrins all, α2β1, and α3β1 bind to Col1 (Ruoslahti, 1991; Takada et al., 2007). This disclosure shows that Col1 homotrimers can interact with integrin α3β1 and persistently induce pro-survival signals. Moreover, suppression of DDR1 leads to the upregulation of integrin α3β1. Further disclosed is a role for Col1 homotrimers in cancer suppression of T cell infiltration, which may be reversed via reduction or elimination of Col1 homotrimer expression in cancer cells. Collectively, these studies suggest that Col1 homotrimers interact with integrin α3β1 on early stage pancreatic cancer-initiating cells to induce proliferation and survival and to suppress immune cell infiltration. Inhibition of FAK at early stage of pancreatic cancer in KPPC mice leads to significant disease control, validating the importance of this signaling axis in initiation and progression of PDAC. In summary, these studies identify a novel bi-modal contribution of Col1 in PDAC progression with the identification of a novel oncogenic variant of Col1 with implication for development of new therapeutic strategies.

As such, it is contemplated that interfering with the binding of homotrimers to α3β1 integrin using, for example, antibodies, small molecules, siRNAs, anti-sense oligos, CAR-T cells, CAR-NK cells, bispecific antibodies may be used to treat cancer or fibrosis. The inventors have identified several avenues through which to take advantage of this distinction in order to treat cancer. These include (1) the use of antibodies that specifically bind α3β1 integrin; (2) the use of agents to inhibit signaling through α3β1 integrin; (3) siRNA or anti-sense oligos to inhibit expression of α3β1 integrin; (4) the use of CAR-T cells that target α3β1 integrin; (5) the use of CAR-NK cells that target α3β1 integrin; and (6) the use of bispecific antibodies that target both α3β1 integrin and CD3 to direct T cells to the cancer cells. Further contemplated are methods comprising use of such methods for interfering with binding of Col1 homotrimers to α3β1 integrin in combination with immunotherapy, such as checkpoint blockade therapy (e.g., anti-PD-1 therapy).

### I. Antibodies and Production Thereof

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

B. General Methods

It will be understood that monoclonal antibodies binding to α3β1 integrin will have several applications. These include the production of diagnostic kits for use in detecting and diagnosing cancer, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Example carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Example adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce cancer-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art. HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described, and those using polyethylene glycol (PEG), such as 37% (v/v) PEG. The use of electrically induced fusion methods also is appropriate and there are processes for better efficiency. Fusion procedures usually produce viable hybrids at low frequencies, about 1×10⁻⁶ to 1×10⁻⁸, but with optimized procedures one can achieve fusion efficiencies close to 1 in 200. However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Example agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

Example selection media is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

C. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g. a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Here, in some embodiments, the epitope is a linear or conformational epitope that is present in α3β1 integrin.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see U.S. 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, the antibodies may be defined by their variable sequence, which include additional “framework” regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below).

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1-6) Dextran Increases Its Affinity For Antigen,” J. Exp. Med. 168(3): 1099-1109; Tao, M. H. et al. (1989) “Studies Of Aglycosylated Chimeric Mouse-Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

D. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for treatment of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1 mW) outperforms several other nucleoside modifications and their combinations in terms of translation capacity. In addition to turning off the immune/eIF2α phosphorylation-dependent inhibition of translation, incorporated N1 mW nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described. High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life, including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat. In a further aspect of the disclosure the modifications are M252Y/S254T/T256E.

Additionally, various publications describe methods for obtaining physiologically active molecules whose half-lives are modified either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH₂ domain (according to the IMGT unique numbering for C-domain) were substituted with alanine residues. This modification, also known as “LALA” mutation, abolishes antibody binding to FcγRI, FcγRII and FcγRIIIa. The variant and unmodified recombinant mAbs were compared for their capacity to neutralize and enhance infection by the four dengue virus serotypes. LALA variants retained the same neutralizing activity as unmodified mAb, but were completely devoid of enhancing activity. LALA mutations of this nature are therefore contemplated in the context of the presently disclosed antibodies.

Altered Glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)—N-acetylglucosaminyltransferase III (GnT III), such that GnT III adds GlcNAc to the IL-23p19 antibody. Methods for producing antibodies in such a fashion are provided in WO/9954342, WO/03011878, patent publication U.S. 2003/0003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cutoff. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection; however it has become clear that many human naturally occurring antibodies with autoreactive properties persist in adult mature repertoires. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

E. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Ga14 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane. The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

F. Multispecific Antibodies

In certain embodiments, antibodies of the present disclosure are bispecific or multispecific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an antigen-specific arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess an antigen-binding arm and an arm that binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′).sub.2 bispecific antibodies). WO 96/16673 describes a bispecific anti-ErbB2/anti-Fc gamma RIII antibody and U.S. Pat. No. 5,837,234 discloses a bispecific anti-ErbB2/anti-Fc gamma RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full-length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998)). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

In a particular embodiment, a bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). A multivalent antibody may be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1—(X1).sub.n-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH—CH1-flexible linker-VH—CH1-Fc region chain; or VH—CH1—VH—CH1-Fc region chain. The multivalent antibody herein preferably further comprises at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

G. Chimeric Antigen Receptors

Chimeric antigen receptor molecules are recombinant fusion protein and are distinguished by their ability to both bind antigen and transduce activation signals via immunoreceptor activation motifs (ITAMs) present in their cytoplasmic tails. Receptor constructs utilizing an antigen-binding moiety (for example, generated from single chain antibodies (scFv) afford the additional advantage of being “universal” in that they bind native antigen on the target cell surface in an HLA-independent fashion.

A chimeric antigen receptor can be produced by any means known in the art, though preferably it is produced using recombinant DNA techniques. A nucleic acid sequence encoding the several regions of the chimeric antigen receptor can be prepared and assembled into a complete coding sequence by standard techniques of molecular cloning (genomic library screening, PCR, primer-assisted ligation, scFv libraries from yeast and bacteria, site-directed mutagenesis, etc.). The resulting coding region can be inserted into an expression vector and used to transform a suitable expression host allogeneic or autologous immune effector cells, such as a T cell or an NK cell.

Embodiments of the CARs described herein include nucleic acids encoding an antigen-specific chimeric antigen receptor (CAR) polypeptide, including a comprising an intracellular signaling domain, a transmembrane domain, and an extracellular domain comprising one or more signaling motifs. In certain embodiments, the CAR may recognize an epitope comprised of the shared space between one or more antigens. In some embodiments, the chimeric antigen receptor comprises: a) an intracellular signaling domain, b) a transmembrane domain, and c) an extracellular domain comprising an antigen binding domain. Optionally, a CAR can comprise a hinge domain positioned between the transmembrane domain and the antigen binding domain. In certain aspects, a CAR of the embodiments further comprises a signal peptide that directs expression of the CAR to the cell surface. For example, in some aspects, a CAR can comprise a signal peptide from GM-CSF.

In certain embodiments, the CAR can also be co-expressed with a membrane-bound cytokine to improve persistence when there is a low amount of tumor-associated antigen. For example, CAR can be co-expressed with membrane-bound IL-15.

Depending on the arrangement of the domains of the CAR and the specific sequences used in the domains, immune effector cells expressing the CAR may have different levels activity against target cells. In some aspects, different CAR sequences may be introduced into immune effector cells to generate engineered cells, the engineered cells selected for elevated SRC and the selected cells tested for activity to identify the CAR constructs predicted to have the greatest therapeutic efficacy.

1. Antigen Binding Domain

In certain embodiments, an antigen binding domain can comprise complementary determining regions of a monoclonal antibody, variable regions of a monoclonal antibody, and/or antigen binding fragments thereof. In another embodiment, that specificity is derived from a peptide (e.g., cytokine) that binds to a receptor. A “complementarity determining region (CDR)” is a short amino acid sequence found in the variable domains of antigen receptor (e.g., immunoglobulin and T-cell receptor) proteins that complements an antigen and therefore provides the receptor with its specificity for that particular antigen. Each polypeptide chain of an antigen receptor contains three CDRs (CDR1, CDR2, and CDR3). Since the antigen receptors are typically composed of two polypeptide chains, there are six CDRs for each antigen receptor that can come into contact with the antigen—each heavy and light chain contains three CDRs. Because most sequence variation associated with immunoglobulins and T-cell receptors are found in the CDRs, these regions are sometimes referred to as hypervariable domains. Among these, CDR3 shows the greatest variability as it is encoded by a recombination of the VJ (VDJ in the case of heavy chain and TCR αβ chain) regions.

It is contemplated that the CAR nucleic acids, in particular the scFv sequences are human genes to enhance cellular immunotherapy for human patients. In a specific embodiment, there is provided a full length CAR cDNA or coding region. The antigen binding regions or domains can comprise a fragment of the VH and VL chains of a single-chain variable fragment (scFv) derived from a particular mouse, or human or humanized monoclonal antibody. The fragment can also be any number of different antigen binding domains of an antigen-specific antibody. In a more specific embodiment, the fragment is an antigen-specific scFv encoded by a sequence that is optimized for human codon usage for expression in human cells. In certain aspects, VH and VL domains of a CAR are separated by a linker sequence, such as a Whitlow linker. CAR constructs that may be modified or used according to the embodiments are also provided in International (PCT) Patent Publication No. WO/2015/123642, incorporated herein by reference.

As previously described, the prototypical CAR encodes a scFv comprising VH and VL domains derived from one monoclonal antibody (mAb), coupled to a transmembrane domain and one or more cytoplasmic signaling domains (e.g. costimulatory domains and signaling domains). Thus, a CAR may comprise the LCDR1-3 sequences and the HCDR1-3 sequences of an antibody that binds to an antigen of interest, such as tumor associated antigen. In further aspects, however, two of more antibodies that bind to an antigen of interest are identified and a CAR is constructed that comprises: (1) the HCDR1-3 sequences of a first antibody that binds to the antigen; and (2) the LCDR1-3 sequences of a second antibody that binds to the antigen. Such a CAR that comprises HCDR and LCDR sequences from two different antigen binding antibodies may have the advantage of preferential binding to particular conformations of an antigen (e.g., conformations preferentially associated with cancer cells versus normal tissue).

Alternatively, it is shown that a CAR may be engineered using VH and VL chains derived from different mAbs to generate a panel of CAR+ T cells. The antigen binding domain of a CAR can contain any combination of the LCDR1-3 sequences of a first antibody and the HCDR1-3 sequences of a second antibody.

2. Hinge Domain

In certain aspects, a CAR polypeptide of the embodiments can include a hinge domain positioned between the antigen binding domain and the transmembrane domain. In some cases, a hinge domain may be included in CAR polypeptides to provide adequate distance between the antigen binding domain and the cell surface or to alleviate possible steric hindrance that could adversely affect antigen binding or effector function of CAR-gene modified T cells. In some aspects, the hinge domain comprises a sequence that binds to an Fc receptor, such as FcγR2a or FcγR1a. For example, the hinge sequence may comprise an Fc domain from a human immunoglobulin (e.g., IgG1, IgG2, IgG3, IgG4, IgA1, IgA2, IgM, IgD or IgE) that binds to an Fc receptor. In certain aspects, the hinge domain (and/or the CAR) does not comprise a wild type human IgG4 CH2 and CH3 sequence.

In some cases the CAR hinge domain could be derived from human immunoglobulin (Ig) constant region or a portion thereof including the Ig hinge, or from human CD8 α transmembrane domain and CD8a-hinge region. In one aspect, the CAR hinge domain can comprise a hinge-CH₂—CH₃ region of antibody isotype IgG₄. In some aspects, point mutations could be introduced in antibody heavy chain CH₂ domain to reduce glycosylation and non-specific Fc gamma receptor binding of CAR-T cells or any other CAR-modified cells.

In certain aspects, a CAR hinge domain of the embodiments comprises an Ig Fc domain that comprises at least one mutation relative to wild type Ig Fc domain that reduces Fc-receptor binding. For example, the CAR hinge domain can comprise an IgG4-Fc domain that comprises at least one mutation relative to wild type IgG4-Fc domain that reduces Fc-receptor binding. In some aspects, a CAR hinge domain comprises an IgG4-Fc domain having a mutation (such as an amino acid deletion or substitution) at a position corresponding to L235 and/or N297 relative to the wild type IgG4-Fc sequence. For example, a CAR hinge domain can comprise an IgG4-Fc domain having a L235E and/or a N297Q mutation relative to the wild type IgG4-Fc sequence. In further aspects, a CAR hinge domain can comprise an IgG4-Fc domain having an amino acid substitution at position L235 for an amino acid that is hydrophilic, such as R, H, K, D, E, S, T, N or Q or that has similar properties to an “E” such as D. In certain aspects, a CAR hinge domain can comprise an IgG4-Fc domain having an amino acid substitution at position N297 for an amino acid that has similar properties to a “Q” such as S or T.

In certain specific aspects, the hinge domain comprises a sequence that is about 85%, 90%, 91%, 92%, 93%, 94%, 95%, 96%, 97%, 98%, 99% or 100% identical to an IgG4 hinge domain, a CD8a hinge domain, a CD28 hinge domain or an engineered hinge domain.

3. Transmembrane Domain

The antigen-specific extracellular domain and the intracellular signaling-domain may be linked by a transmembrane domain. Polypeptide sequences that can be used as part of transmembrane domain include, without limitation, the human CD4 transmembrane domain, the human CD28 transmembrane domain, the transmembrane human CD3ζ domain, or a cysteine mutated human CD3ζ domain, or other transmembrane domains from other human transmembrane signaling proteins, such as CD16 and CD8 and erythropoietin receptor. In some aspects, for example, the transmembrane domain comprises a sequence at least 85%, 90%, 91%, 92%, 93%, 94%, 95%, 96%, 97%, 98%, 99% or 100% identical to one of those provided in U.S. Patent Publication No. 2014/0274909 (e.g. a CD8 and/or a CD28 transmembrane domain) or U.S. Pat. No. 8,906,682 (e.g. a CD8α transmembrane domain), both incorporated herein by reference. Transmembrane regions of particular use in this invention may be derived from (i.e. comprise at least the transmembrane region(s) of) the alpha, beta or zeta chain of the T-cell receptor, CD3 epsilon, CD45, CD4, CD5, CD8, CD9, CD16, CD22, CD33, CD37, CD64, CD80, CD86, CD134, CD137, CD154. In certain specific aspects, the transmembrane domain can be 85%, 90%, 91%, 92%, 93%, 94%, 95%, 96%, 97%, 98%, 99% or 100% identical to a CD8a transmembrane domain or a CD28 transmembrane domain.

4. Intracellular Signaling Domain

The intracellular signaling domain of the chimeric antigen receptor of the embodiments is responsible for activation of at least one of the normal effector functions of the immune cell engineered to express a chimeric antigen receptor. The term “effector function” refers to a specialized function of a differentiated cell. Effector function of a T cell, for example, may be cytolytic activity or helper activity including the secretion of cytokines. Effector function in a naive, memory, or memory-type T cell includes antigen-dependent proliferation. Thus the term “intracellular signaling domain” refers to the portion of a protein that transduces the effector function signal and directs the cell to perform a specialized function. In some aspects, the intracellular signaling domain is derived from the intracellular signaling domain of a native receptor. Examples of such native receptors include the zeta chain of the T-cell receptor or any of its homologs (e.g., eta, delta, gamma, or epsilon), MB1 chain, B29, Fc RIII, Fc RI, and combinations of signaling molecules, such as CD3ζ and CD28, CD27, 4-1BB, DAP-10, OX40, and combinations thereof, as well as other similar molecules and fragments. Intracellular signaling portions of other members of the families of activating proteins can be used. While usually the entire intracellular signaling domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term “intracellular signaling domain” is thus meant to include a truncated portion of the intracellular signaling domain sufficient to transduce the effector function signal, upon CAR binding to a target. In a preferred embodiment, the human CD3ζ intracellular domain is used as the intracellular signaling domain for a CAR of the embodiments.

In specific embodiments, intracellular receptor signaling domains in the CAR include those of the T cell antigen receptor complex, such as the ζ chain of CD3, also Fcγ RIII costimulatory signaling domains, CD28, CD27, DAP10, CD137, OX40, CD2, alone or in a series with CD3ζ, for example. In specific embodiments, the intracellular domain (which may be referred to as the cytoplasmic domain) comprises part or all of one or more of TCRζ chain, CD28, CD27, OX40/CD134, 4-1BB/CD137, FcεRIγ, ICOS/CD278, IL-2Rβ/CD122, IL-2Rα/CD132, DAP10, DAP12, and CD40. In some embodiments, one employs any part of the endogenous T cell receptor complex in the intracellular domain. One or multiple cytoplasmic domains may be employed, as so-called third generation CARs have at least two or three signaling domains fused together for additive or synergistic effect, for example the CD28 and 4-1BB can be combined in a CAR construct.

In some embodiments, the CAR comprises additional other costimulatory domains. Other costimulatory domains can include, but are not limited to one or more of CD28, CD27, OX-40 (CD134), DAP10, and 4-1BB (CD137). In addition to a primary signal initiated by CD3ζ, an additional signal provided by a human costimulatory receptor inserted in a human CAR is important for full activation of T cells and could help improve in vivo persistence and the therapeutic success of the adoptive immunotherapy.

In certain specific aspects, the intracellular signaling domain comprises a sequence 85%, 90%, 91%, 92%, 93%, 94%, 95%, 96%, 97%, 98%, 99% or 100% identical to a CD3ζ intracellular domain, a CD28 intracellular domain, a CD137 intracellular domain, or a domain comprising a CD28 intracellular domain fused to the 4-1BB intracellular domain.

H. ADCs

Antibody Drug Conjugates or ADCs are a new class of highly potent biopharmaceutical drugs designed as a targeted therapy for the treatment of people with disease. ADCs are complex molecules composed of an antibody (a whole mAb or an antibody fragment such as a single-chain variable fragment, or scFv) linked, via a stable chemical linker with labile bonds, to a biological active cytotoxic/anti-viral payload or drug. Antibody Drug Conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the diseased cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on infected cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cell or impairs cellular replication. Due to this targeting, ideally the drug has lower side effects and gives a wider therapeutic window than other agents.

A stable link between the antibody and cytotoxic agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (anti-cancer) drug. For example, a non-cleavable linker keeps the drug within the cell. As a result, the entire antibody, linker and cytotoxic agent enter the targeted cancer cell where the antibody is degraded to the level of an amino acid. The resulting complex—amino acid, linker and cytotoxic agent—now becomes the active drug. In contrast, cleavable linkers are catalyzed by enzymes in the host cell where it releases the cytotoxic agent.

Another type of cleavable linker, currently in development, adds an extra molecule between the cytotoxic drug and the cleavage site. This linker technology allows researchers to create ADCs with more flexibility without worrying about changing cleavage kinetics. Researchers are also developing a new method of peptide cleavage based on Edman degradation, a method of sequencing amino acids in a peptide. Future direction in the development of ADCs also include the development of site-specific conjugation (TDCs) to further improve stability and therapeutic index and a emitting immunoconjugates and antibody-conjugated nanoparticles.

I. BiTES

Bi-specific T-cell engagers (BiTEs) are a class of artificial bispecific monoclonal antibodies that are investigated for the use as anti-cancer drugs. They direct a host's immune system, more specifically the T cells' cytotoxic activity, against infected cells. BiTE is a registered trademark of Micromet AG.

BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic activity on infected cells by producing proteins like perforin and granzymes, independently of the presence of MHC I or co-stimulatory molecules. These proteins enter infected cells and initiate the cell's apoptosis. This action mimics physiological processes observed during T cell attacks against infected cells.

J. Intrabodies

In a particular embodiment, the antibody is a recombinant antibody that is suitable for action inside of a cell—such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. One means of delivery comprises the use of lipid-based nanoparticles, or exosomes, as taught in U.S. Pat. Appln. Pubin. 2018/0177727, which is incorporated by reference here in its entirety. With respect to the stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.).

K. Purification

The antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

L. Antibody Conjugates

Antibodies of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ⁵²Eu, gallium⁶⁷, ³hydrogen, iodine¹²³, iodine ¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetraacetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light. In particular, 2- and 8-azido analogues of purine nucleotides have been used as site-directed photoprobes to identify nucleotide binding proteins in crude cell extracts. The 2- and 8-azido nucleotides have also been used to map nucleotide binding domains of purified proteins and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature. This approach has been reported to produce diagnostically and therapeutically promising antibodies which are currently in clinical evaluation.

### II. Methods of Treatment

Certain aspects of the present embodiments can be used to prevent or treat a disease or disorder associated with the presence of homotrimeric type I collagen, such as pancreatic ductal adenocarcinoma (PDAC). Functioning of homotrimeric type I collagen may be reduced by any suitable drugs that disrupts the interaction between homotrimeric type I collagen and α3β1 integrin. For example, such substances could include an anti-031 integrin antibody, an α3 integrin siRNA, a Col1 siRNA, etc.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that targets α3β1 integrin either alone or in combination with administration of chemotherapy, immunotherapy, or radiotherapy, performance of surgery, or any combination thereof.

The term “subject” as used herein refers to any individual or patient to which the subject methods are performed. Generally, the subject is human, although as will be appreciated by those in the art, the subject may be an animal. Thus, other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease, such as a cancer or a fibroid disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, a neurodegenerative disease, and/or a genetic disorder).

B. Formulation and Administration

The present disclosure provides pharmaceutical compositions comprising antibodies that selectively bind to α3β1 integrin. Such compositions comprise a prophylactically or therapeutically effective amount of an antibody or a fragment thereof and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Passive transfer of antibodies generally will involve the use of intravenous or intramuscular injections. The forms of antibody can be as monoclonal antibodies (MAb). Such immunity generally lasts for only a short period of time, and there is also a potential risk for hypersensitivity reactions, and serum sickness, especially from gamma globulin of non-human origin. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

C. Kits and Diagnostics

In various aspects of the embodiments, a kit is envisioned containing therapeutic agents and/or other therapeutic and delivery agents. The present embodiments contemplate a kit for preparing and/or administering a therapy of the embodiments. The kit may comprise one or more sealed vials containing any of the pharmaceutical compositions of the present embodiments. The kit may include, for example, at least one α3β1 integrin antibody as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

D. ADCC

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. By “antibody having increased/reduced antibody dependent cell-mediated cytotoxicity (ADCC)” is meant an antibody having increased/reduced ADCC as determined by any suitable method known to those of ordinary skill in the art.

As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example, the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

E. CDC

Complement-dependent cytotoxicity (CDC) is a function of the complement system. It is the processes in the immune system that kill pathogens by damaging their membranes without the involvement of antibodies or cells of the immune system. There are three main processes. All three insert one or more membrane attack complexes (MAC) into the pathogen which cause lethal colloid-osmotic swelling, i.e., CDC. It is one of the mechanisms by which antibodies or antibody fragments have a cytotoxic effect.

F. Combination Therapy

In certain embodiments, the compositions and methods of the present embodiments involve an antibody or an antibody fragment against α3β1 integrin to inhibit its activity, in combination with a second or additional therapy, such as chemotherapy or immunotherapy (e.g., checkpoint blockade therapy). Such therapy can be applied in the treatment of any disease that is associated with elevated homotrimeric type I collagen. For example, the disease may be a cancer or a fibroid disease.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

A therapeutic antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an antibody therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

2. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredepa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids, such as retinoic acid; capecitabine; carboplatin, procarbazine, plicomycin, gemcitabine, navelbine, farnesyl-protein transferase inhibitors, transplatinum, and pharmaceutically acceptable salts, acids, or derivatives of any of the above.

3. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

4. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169); cytokine therapy, e.g., interferons α, β, and γ, IL-1, GM-CSF, and TNF; gene therapy, e.g., TNF, IL-1, IL-2, and p53 (U.S. Pat. Nos. 5,830,880 and 5,846,945); and monoclonal antibodies, e.g., anti-CD20, anti-ganglioside GM2, and anti-p185 (U.S. Pat. No. 5,824,311). It is contemplated that one or more anti-cancer therapies may be employed with the antibody therapies described herein.

In some embodiments, the immunotherapy may be comprise an immune checkpoint inhibitor (i.e., may be a checkpoint blockade therapy). Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular embodiments, the immune checkpoint inhibitors target the PD-1 axis and/or CTLA-4. In some embodiments, an immunotherapy of the present disclosure comprises an anti-PD-1 checkpoint blockade therapy (e.g., an anti-PD-1 antibody).

The immune checkpoint inhibitors may be drugs such as small molecules, recombinant forms of ligand or receptors, or, in particular, may be antibodies, such as human antibodies (e.g., International Patent Publication WO2015016718, incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering (Park, Rosenberg et al. 2011). Isolation and transfer of tumor specific T cells has been shown to be successful in treating melanoma. Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3 zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

5. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1—Targeting Alpha3 Beta1 (α3β1) Integrin for Treatment of Cancer

Materials and Methods

Histology and immunohistochemistry. For paraffin-fixed samples, mouse tissues were fixed in 10% neutral buffered formalin, embedded in paraffin, and sectioned at 5 μm thickness. Sections were processed for hematoxylin and eosin (H&E) staining. Masson's trichrome stain (MTS) was conducted using Gomori's Trichrome Stain Kit (38016SS2, Leica Biosystems). Picrosirius red staining for collagen was conducted using 0.1% Picrosirius Red (Direct Red80; Sigma) and counterstained with Weigert's haematoxylin. Images were captured with a Leica DM 1000 LED microscope and an MC120 HD Microscope Camera with Las V4.4 Software (Leica). Formalin-fixed, paraffin-embedded sections were processed for immunohistochemical staining as previously documented (Chen et al., 2018). Sections were incubated with primary antibodies: αSMA (M0851, Dako, 1:100), CK19 (ab52625, Abcam, 1:200), collagen I (ab34710, Abcam, 1:200), integrin α3 (ab131055, Abcam, 1:300), Sox9 (ab185966, Abcam, 1:200), then biotinylated secondary antibodies, and streptavidin HRP (Biocare Medical). For all immunolabeling experiments, sections were developed by DAB and counterstained with hematoxylin.

Single cell RNA sequencing (sc-RNA-seq). Fresh tumor tissue of a KPPC mouse was minced with sterilized lancets, digested with collagenase IV (17104019, Gibco, 4 mg/mL)/dispase II (17105041, Gibco, 4 mg/mL)/DMEM at 37° C. for 0.5 h, filtered by 70 μm cell strainers, and resuspended in PBS/2% FBS as single cell suspension. The single cell suspension was stained with Live/Dead viability dye eFluor 780 (65-0865-14, eBioscience), filtered through a 40 m mesh, and then sorted for live cells with Aria II sorter (BD Biosciences) at the South Campus Flow Cytometry Core Laboratory of MDACC. For tumors of KPPF (FSF-Kras^{G12D/+}; Trp53frt/frt; Pdx1-Flp) mice, 4064 cells in total from 2 mice were analyzed. For tumors of KPPC (The LSL-Kras^{G12D}; Trp53^{loxP/loxP}; Pdx1-Cre) mice, 3989 cells in total from 2 mice were analyzed. Sc-RNA-seq on these samples was conducted using Chromium Controller and Single Cell 3′ Reagent Kits v2 (10× Genomics) at the Sequencing and Microarray Facility of MDACC. Single cell Gel Bead-In-Emulsions (GEMs) generation and barcoding, post GEM-RT cleanup and cDNA amplification, library construction and Illumina-ready sequencing library generation were prepared by following the manufacturer's guidelines. High Sensitivity dsDNA Qubit kit was used to estimate the cDNA and Library concentration. HS DNA Bioanalyzer was used for the quantification of cDNA. DNA 1000 Bioanalyzer was used for the quantification of libraries. The “c-loupe” files were generated by using Cell Ranger software pipelines following manufacturer's guidelines. Cells from unfractionated tumor were encapsulated using 10× Genomics' Chromium controller and Single Cell 3′ Reagent Kits v2. Following capture and lysis, cDNA was synthesized and amplified to construct Illumina sequencing libraries. The libraries from about 1,000 cells per sample were sequenced with Illumina Nextseq 500. The run format was 26 cycles for read1, 8 cycles index 1, and 124 cycles for read2. sc-RNA-seq data was processed by the Sequencing and Microarray Facility in MD Anderson Cancer Center. Further data analysis was performed by using R package software of the Bioconductor Project.

Western blotting. Extracted type I collagen (Col1) solution and solubilized Matrigel (growth-factor-reduced, 354230, Corning) were prepared in 6×reducing SDS Laemmli Sample Buffer (Bio-world) and denatured at 95° C. for 20 min. Samples were then subjected to electrophoresis using Mini-PROTEAN TGX precast polyacrylamide gels (Bio-Rad) and transblotted onto polyvinylidene fluoride (PVDF) membranes using Trans-Blot Turbo Transfer System (Bio-Rad). Col1 was blotted with goat anti-col1 antibody (1310, SouthernBiotech, 1:1000) and HRP-conjugated donkey anti-goat secondary antibody. Type IV collagen (Co14) was blotted with rabbit anti-Col4 antibody (ab52235, Abcam, 1:300) and HRP-conjugated secondary goat anti-rabbit antibody.

FAK inhibitor (FAKi) treatment in vivo. KPPC mice at the age of 21 days were randomized into control group or FAKi (VS-4718 or PND-1186, Selleckchem) treatment group for further treatment. The mice of FAKi treatment group were treated with 50 mg/kg FAKi resuspended in 0.5% carboxymethyl cellulose (Sigma-Aldrich) and 0.1% Tween-80 (Sigma-Aldrich) in sterile water by oral gavage twice a day, as previously described (Jiang et al., 2016). The mice of control group received vehicle using the same administration strategy. Pancreas tissues from mice were collected and examined at the same age of 28 days after the treatment of 7 days.

Results

Tumors contain both cancer cells and constituents of the tumor microenvironment (TME), such as fibroblasts and type I collagen. It is still unclear if tumor microenvironment serves as a facilitator of tumor growth or restrains tumor growth. There is a possibility that some aspects of the TME can serve as positive regulators of tumor progression and others as negative regulators of tumor growth. Type I collagen (collagen I) produced by the myofibroblasts is a heterotrimer that involves two α1 chains of collagen I (α1(I) collagen) and one α2 chain of collagen I (α2(I) collagen) that is cancer/tumor restraining via binding with potential receptors on cancer cells and other stromal cells (likely discodin domain receptor II-DDR2) and immune cells. In contrast, the cancer cells produce collagen I homotrimers with three α1(I) collagen chains that is cancer/tumor promoting and binds to specific receptors in cancer cells to induce pro-survival signals, anti-apoptotic signals, proliferation signals, and pro-oncogenic signals. The homotrimers (made by cancer cells) are resistant to metalloproteinases and other proteinases when compared to the heterotrimers made by myofibroblasts in the tumor microenvironment. The homotrimers exhibit different structures with the exposure of distinct epitopes compared to heterotrimers, and antibodies generated against the homotrimers will have tumor inhibitory properties by disrupting the signaling through pro-oncogenic receptors on the cancer cells, among other mechanisms.

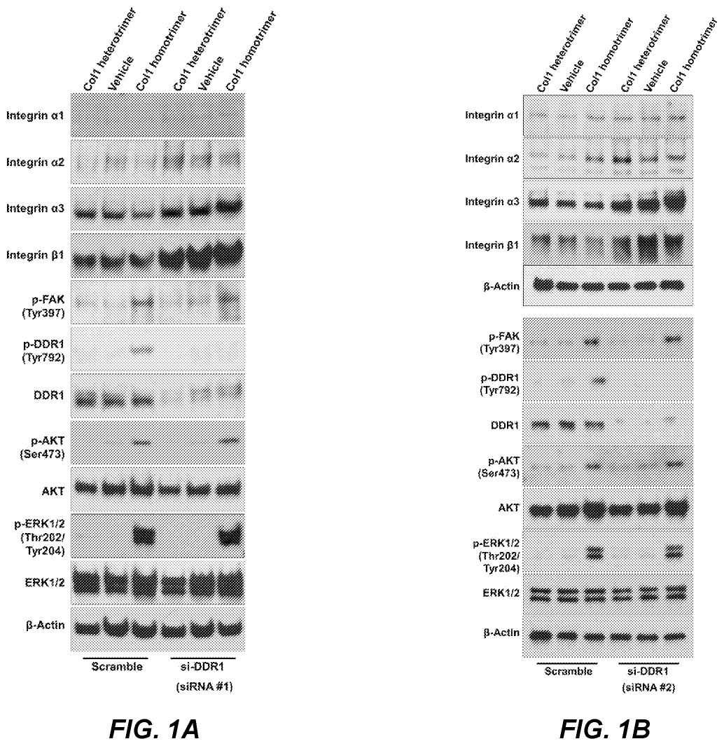

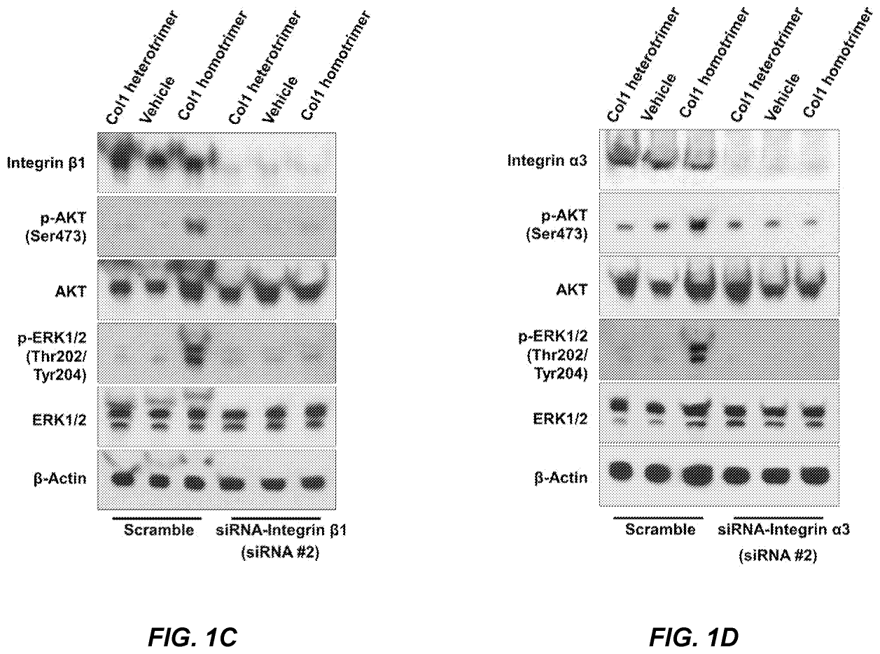

Given that integrins have been widely studied as collagen receptors on cancer cells (Egeblad et al., 2010; Leitinger, 2011; Yeh et al., 2012), the direct functional involvement of integrins in col1 homotrimer-induced pro-survival signaling was examined. Col1 homotrimers induce phosphorylation of DDR1, FAK, AKT and ERK. But knockdown of DDR1 by siRNA did not inhibit the activation of FAK, AKT, and ERK by col1 homotrimers (FIGS. 1A,1B), presumably due to the compensatory upregulation of integrins (especially α3β1) after DDR1 knockdown. Whereas, suppression of integrin β1 by siRNA significantly diminished the col1 homotrimer-induced activation of FAK, AKT, and ERK, suggesting a critical role of integrin β1 in mediating the pro-survival function col1 homotrimers on cancer cells (FIGS. 1C,2A).

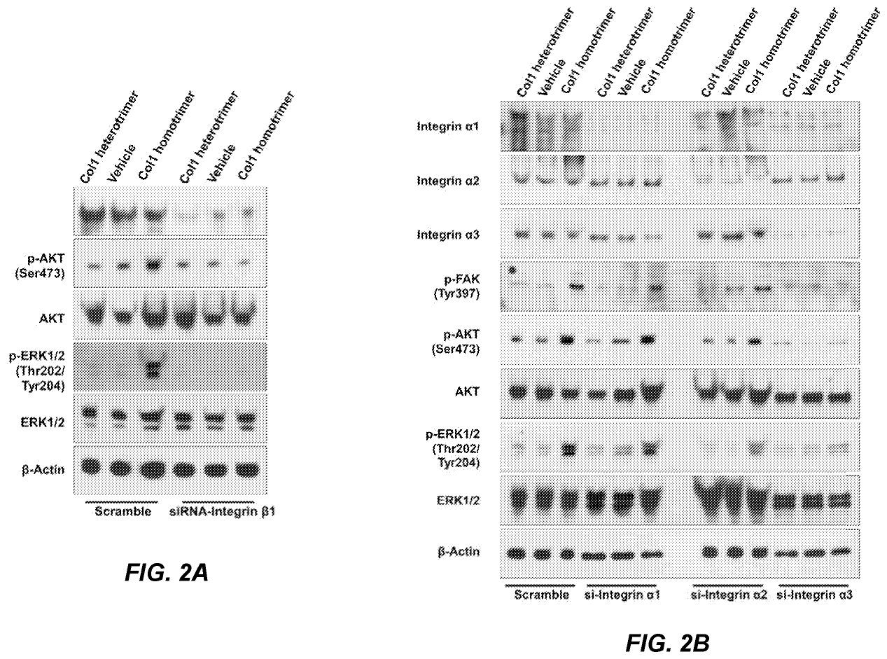

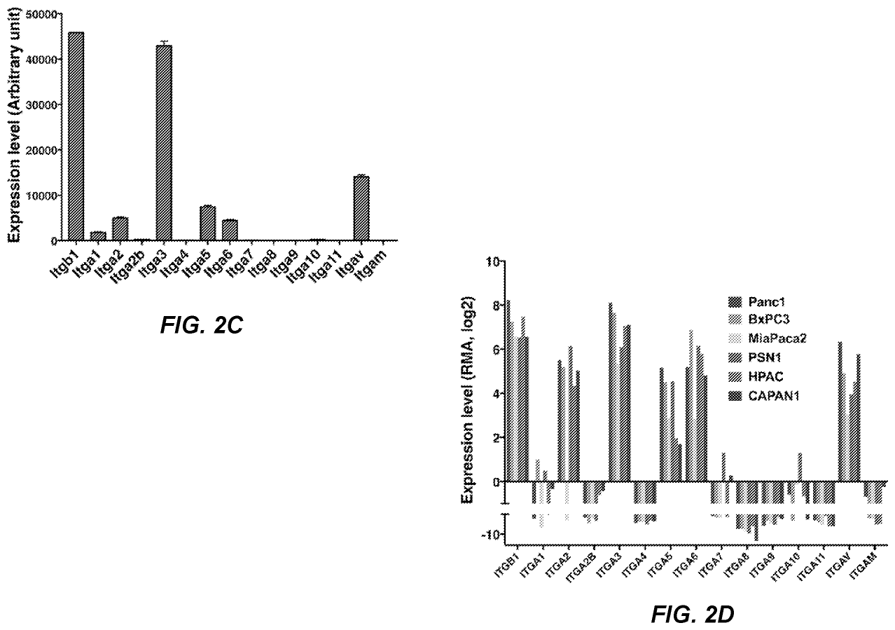

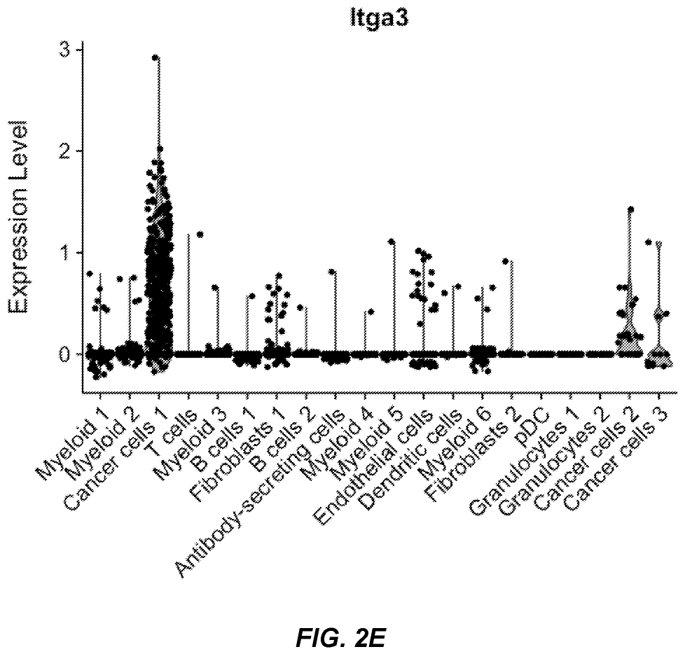

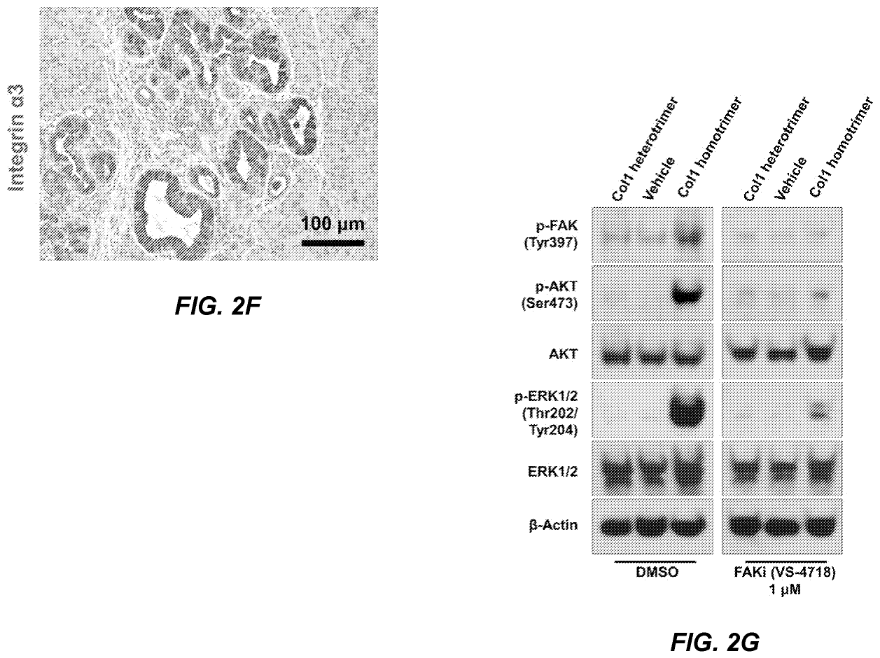

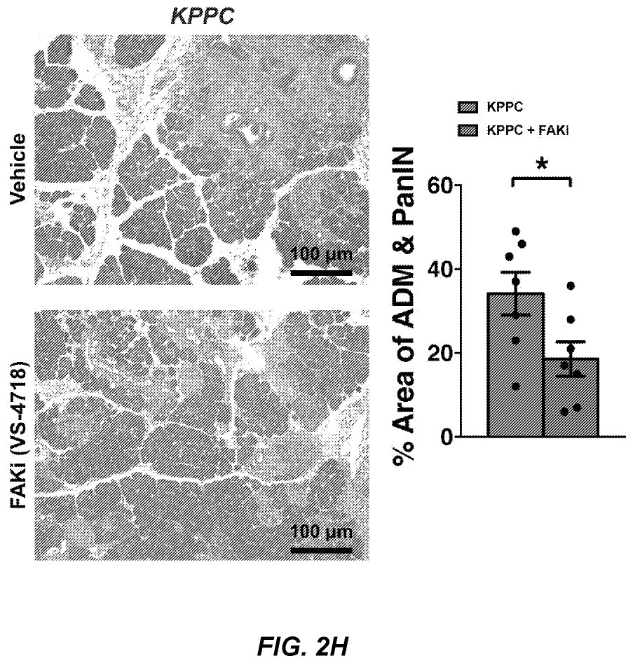

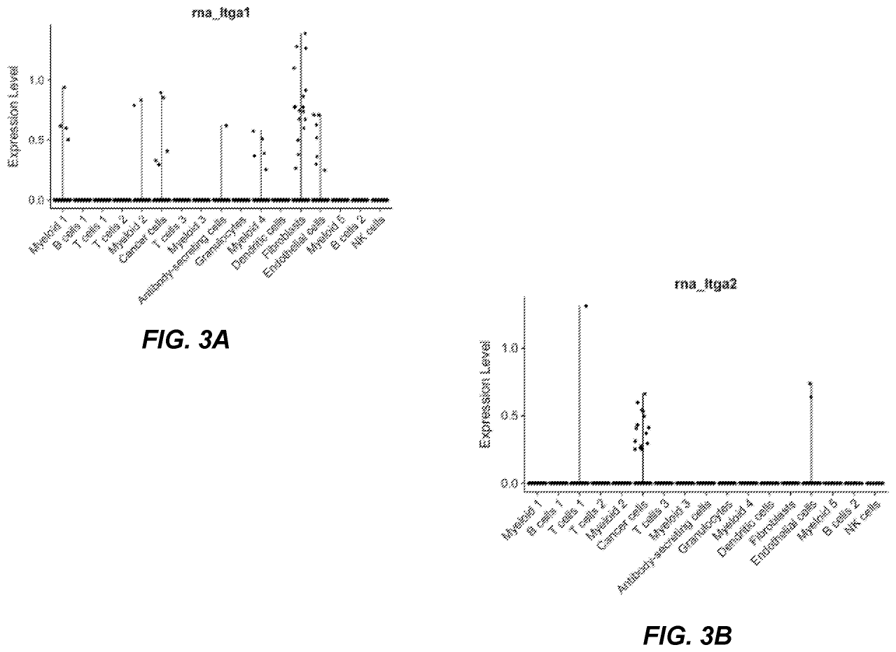

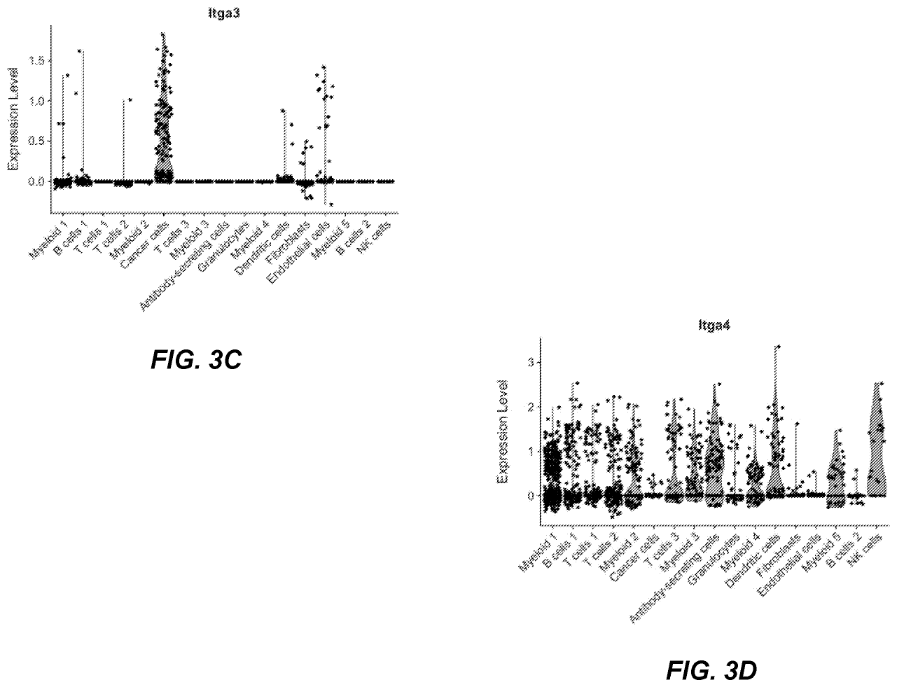

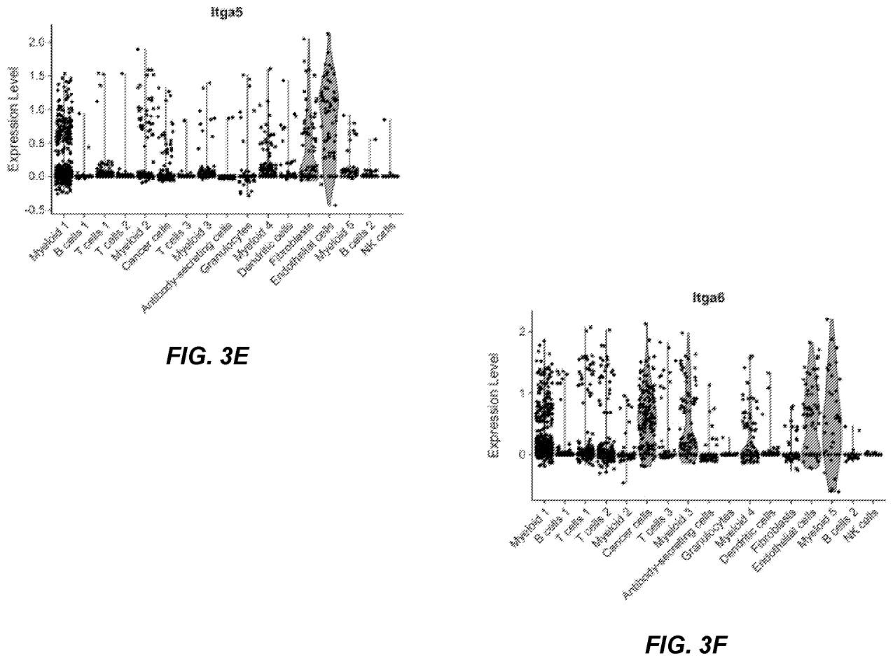

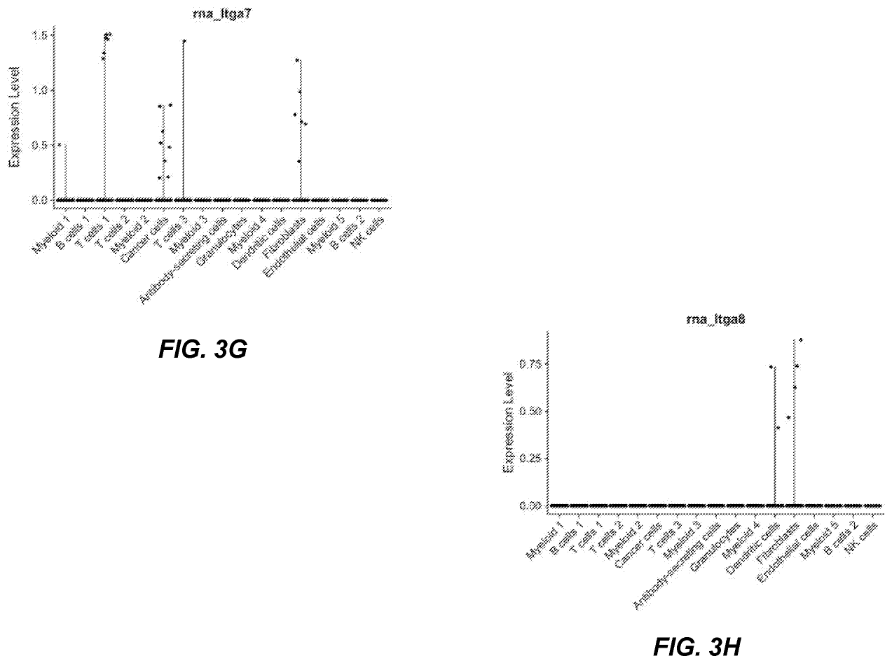

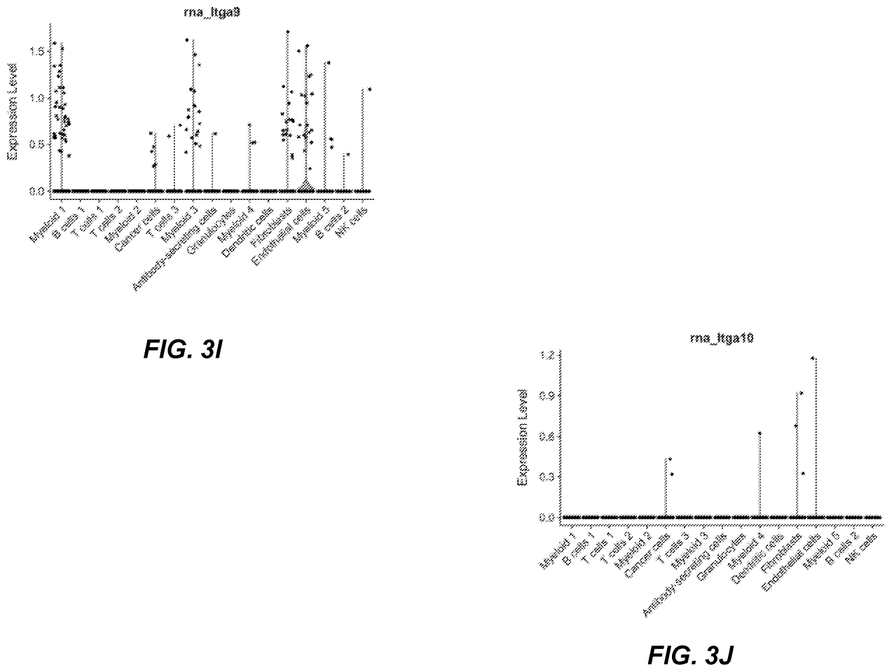

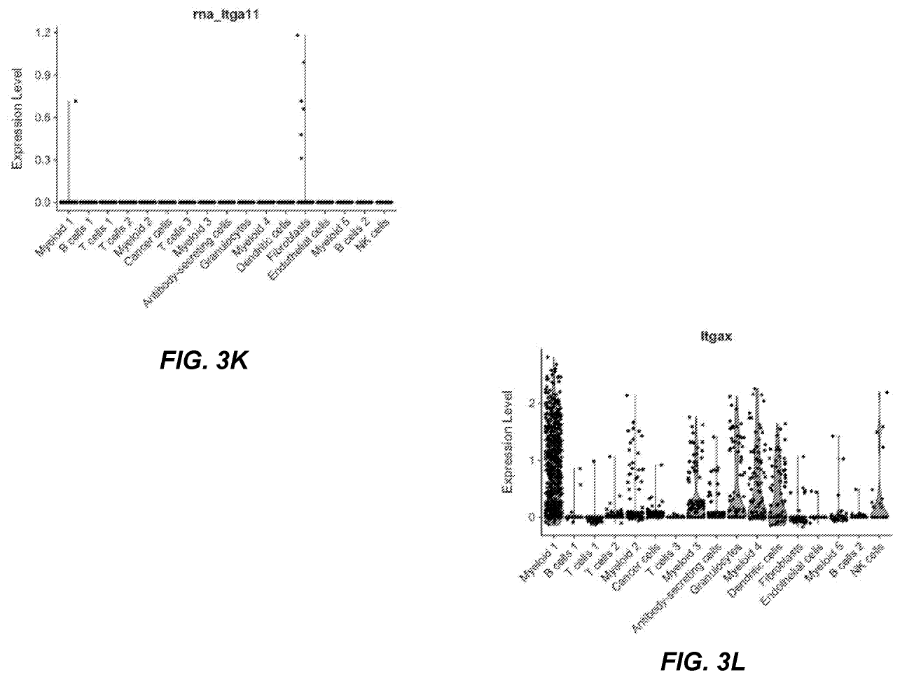

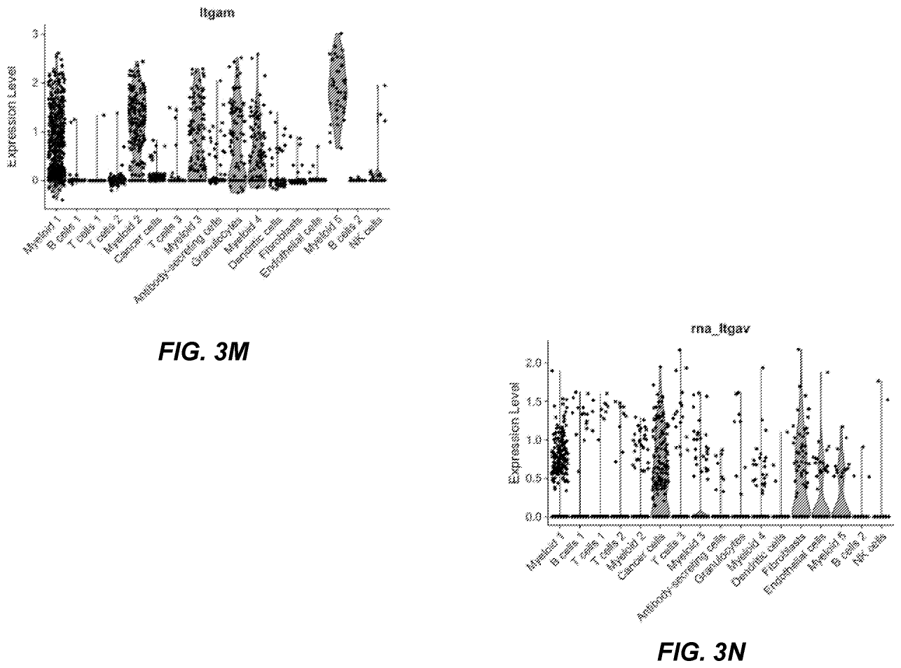

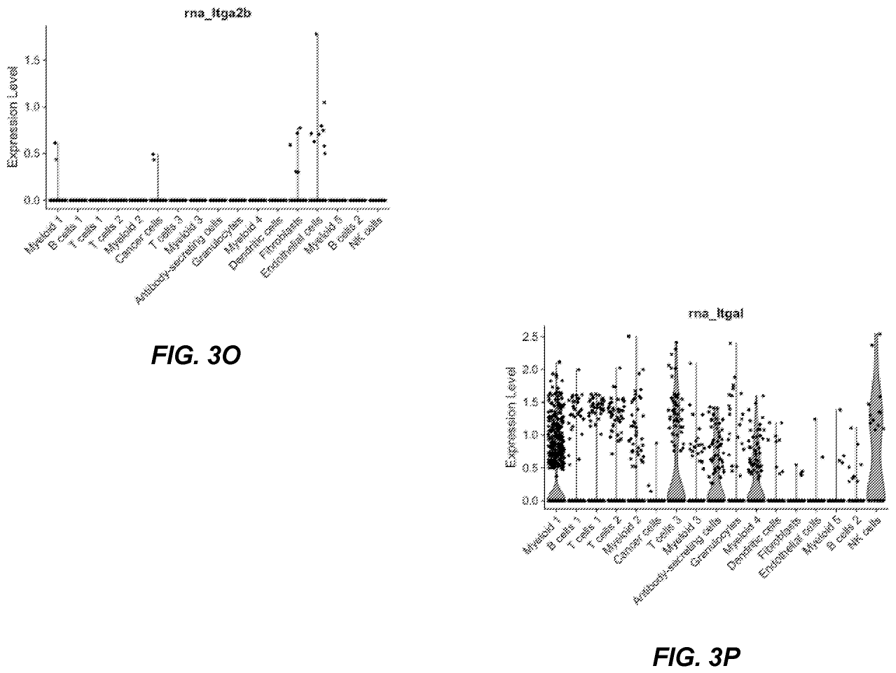

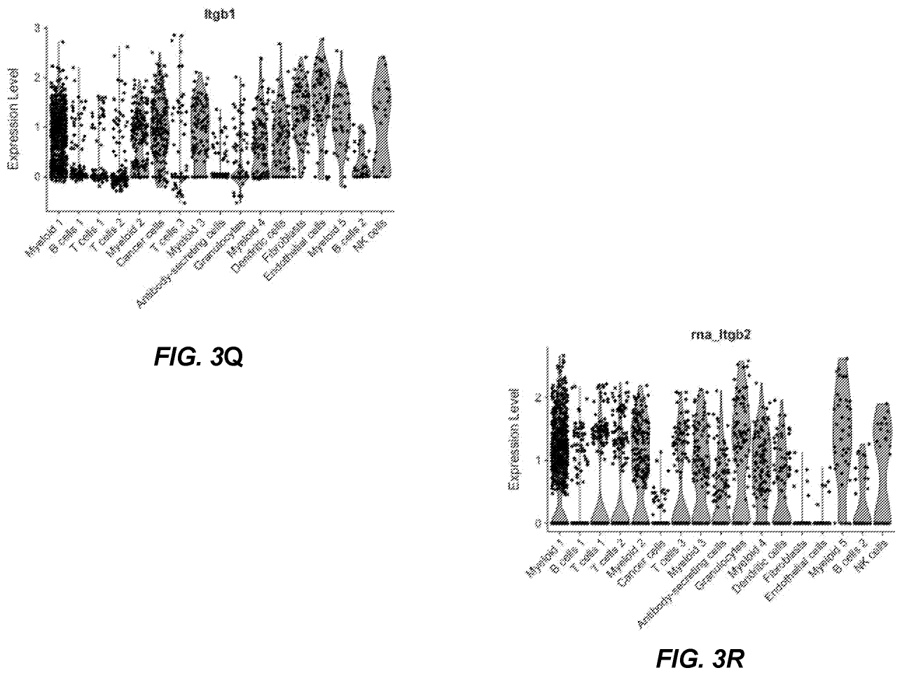

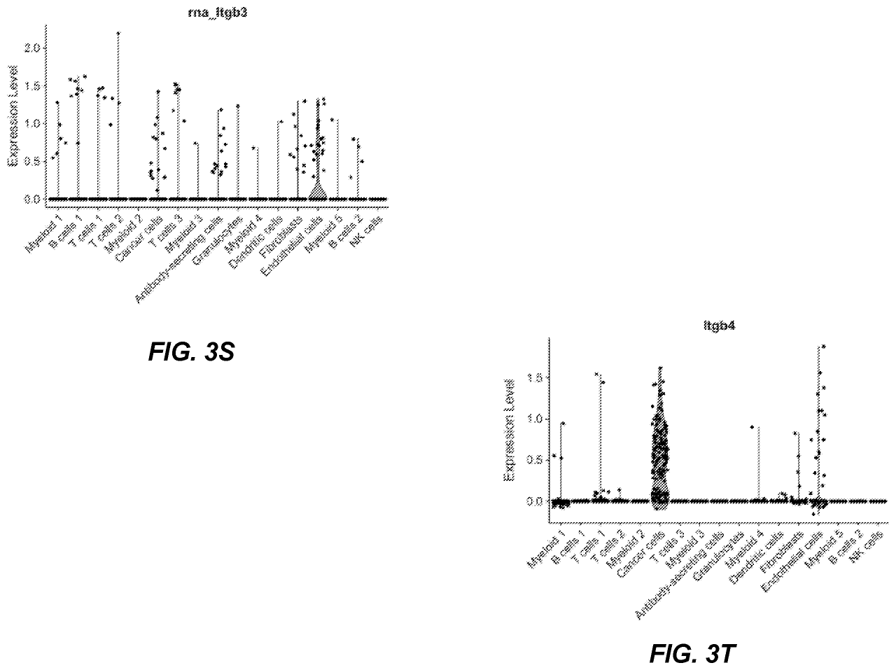

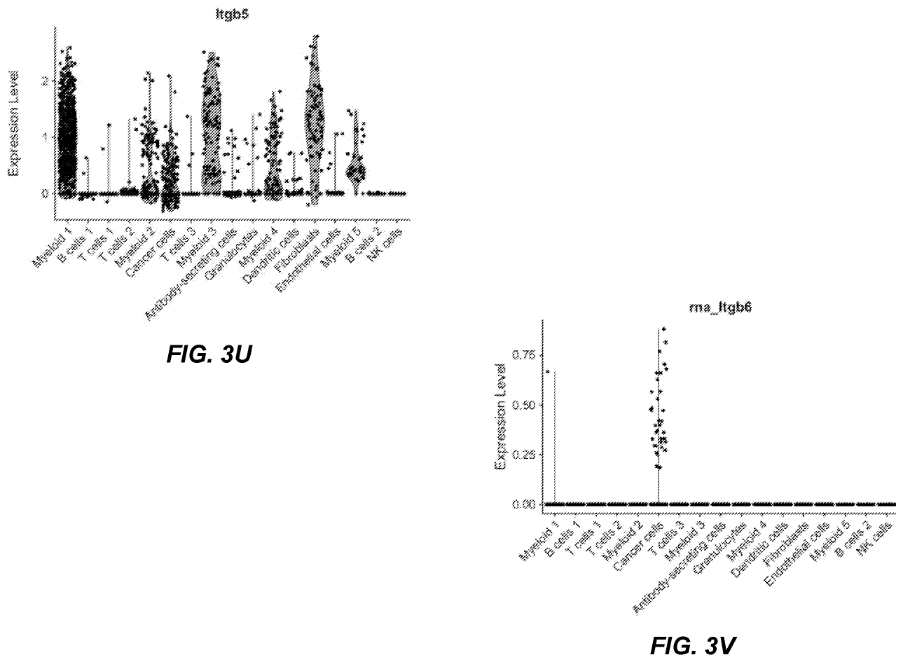

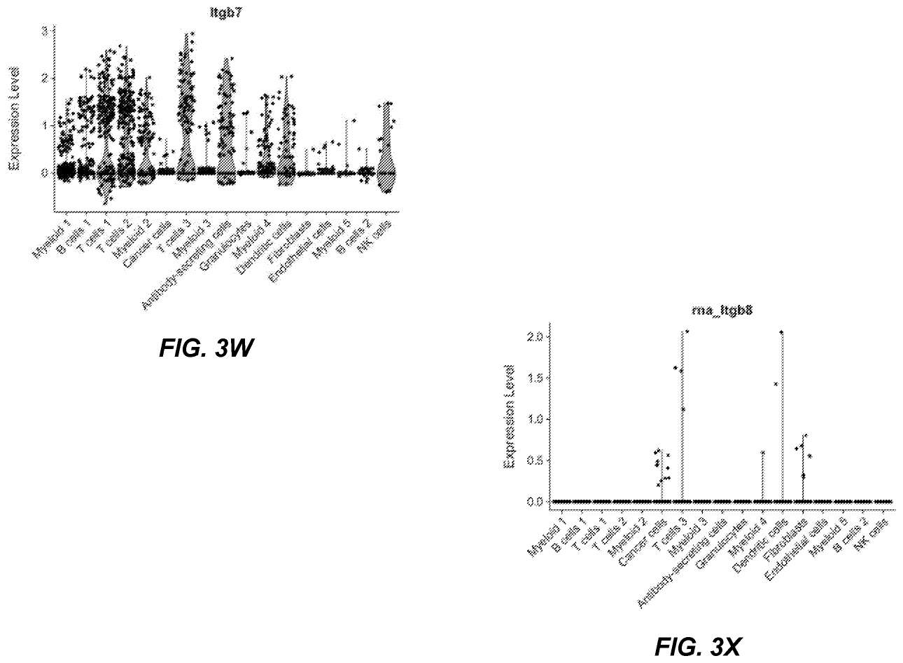

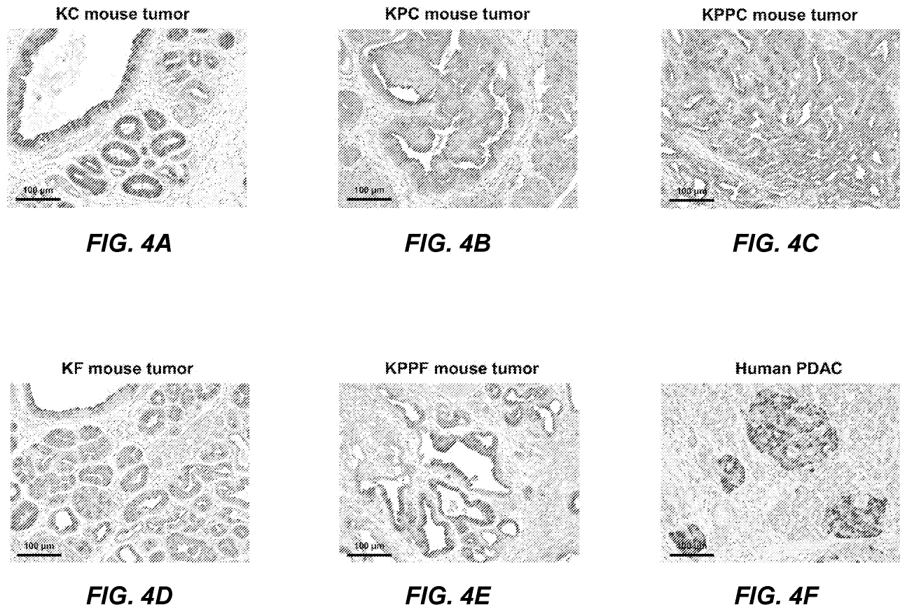

A further examination employing siRNAs against integrin α1, α2, and α3 subunits revealed that siRNAs against integrin α3 (FIG. 1C, 2B) but not α1 integrin subunit significantly inhibits col1 homotrimer-induced phosphorylation of FAK, AKT, and ERK, while siRNA of integrin α2 moderately inhibited col1 homotrimer-induced phosphorylation. RNA-seq data from mouse (FIG. 2C) and human (FIG. 2D, CCLE database) pancreatic cancer cell lines confirms that integrin α3 is the most abundant integrin α-subunit expressed by pancreatic cancer cells. Integrin α10 and α11, also known as collagen-binding integrin subunits along with their 31 integrin subunit partner, revealed minimal expression in pancreatic cancer cells (FIGS. 2C,2D). The extensive expression pattern of integrin α3 in cancer cells was further confirmed by single cell RNA sequencing analysis in KPPC tumors (LSL-Kras^{G12D}; Trp53^{loxP/loxP}; Pdx1-Cre) (FIGS. 2E and 3A-3X) and IHC staining in mouse and human tumors (FIGS. 2F and 4A-4F).

Next, to test the role of col1 homotrimer-induced FAK phosphorylation in the pathogenesis of early lesions associated with pancreatic cancer, a dual inhibitor that targets FAK and PYK2, VS-4718 (PND-1186) was employed and evaluated for its effect on col1 homotrimer-induced pro-survival signaling. VS-4718 inhibited FAK and PYK2 (downstream mediators of integrin- and DDR1-mediated signaling pathways) and abrogated col1 homotrimer induced activation of FAK, AKT, and ERK (FIG. 2G). VS-4718 treatment of KPPC mice also inhibited early PanIN progression (FIG. 2H), consistent with previous observations (Jiang et al., 2016). Taken together, these results establish that col1 homotrimers promotes PDAC cell proliferation by inducing persistent activation of FAK, AKT, and ERK via collagen-binding integrin, c301.

### Example 2—Identification of α3 Integrin Expression in Human PDAC Patients and Correlation with Survival Outcomes

Methods

All human PDAC sections were fixed on tissue microarray slides, which contain three representative 1 mm cores from each patient (two from tumor and one from benign pancreatic tissue). The staining intensity of integrin α3 was quantified by visual scoring of staining on a scale of 0-3 (3—very high, 2—high, 1—low, and 0—negative). The IHC scores of integrin α3 for all samples are graded by combined score of the intensity of staining and the percentage of positive tumor cells. The formula for staining score was used: S=p1×1+p2×2+p3×3, in which p1, p2 and p3 represent fractions of tumor cells representing each staining categories of 1, 2 and 3 respectively. The average score of integrin α3 (ITGA3) expression was 1.87 for the entire cohort. The expression of integrin α3 was categorized as ITGA3-high (n=68) and ITGA3-low (n=62) using the average combined score 1.87 as a cutoff.

Results

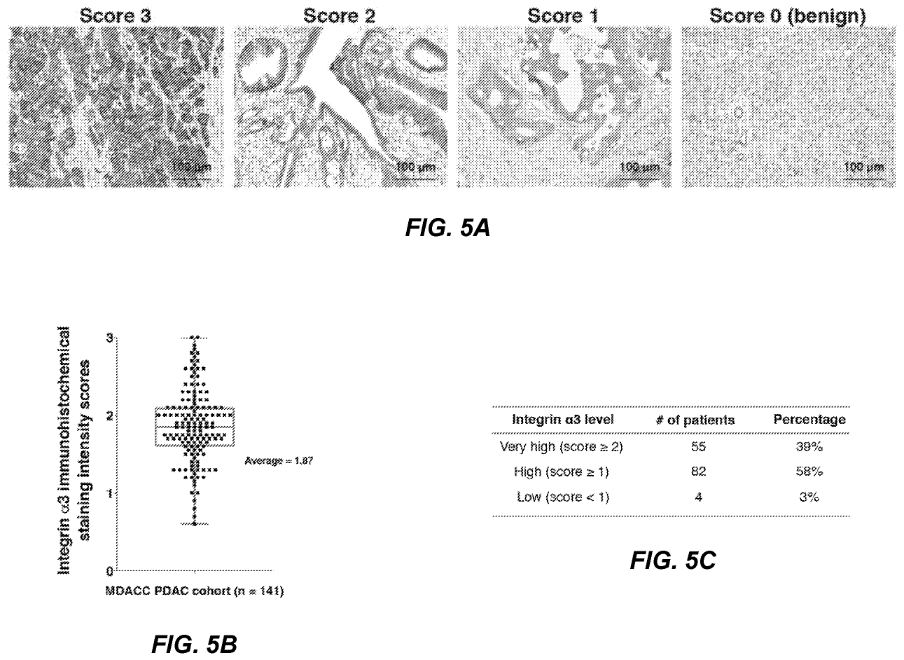

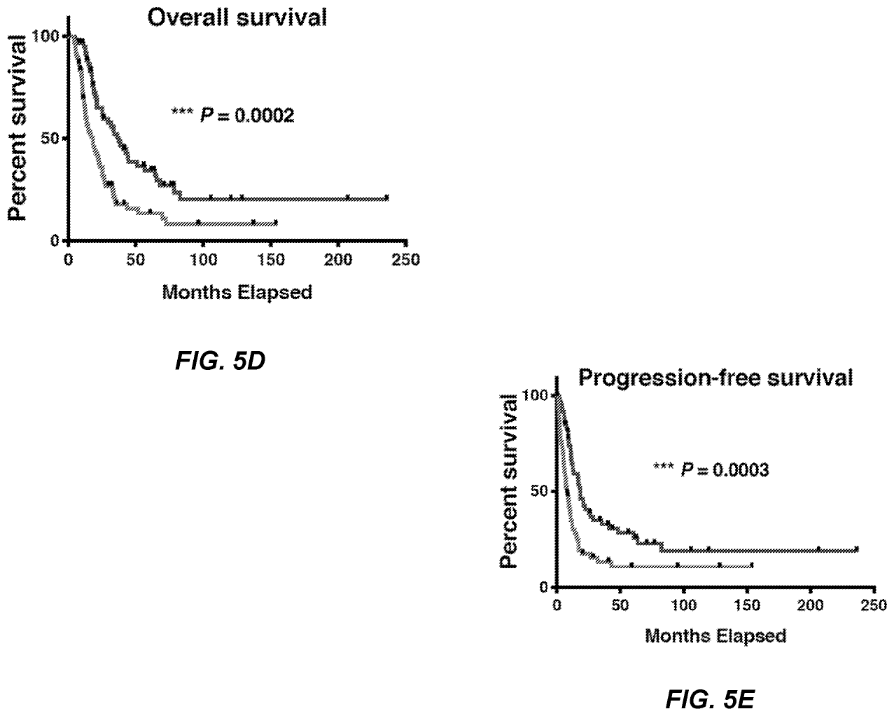

Human PDAC sections (n=141), fixed on tissue microarray slides, were examined for IHC score of integrin α3 (FIG. 5A), showing an average score of 1.87 for the entire cohort (FIG. 5B). The majority (97%) of human PDAC sections revealed very high or high integrin α3 expression (FIG. 5C). High integrin α3 expression level significantly correlated with poor overall survival (FIG. 5D) and progression-free survival (FIG. 5E) of patients. ITGA3-high patients are shown in the lower line of FIGS. 5D and 5E, while ITGA3-low patients are shown in the upper line of FIGS. 5D and 5E.

### Example 3—In Vitro Itga3 siRNA Treatment of PDAC Cells

Methods

KPPC (LSL-Kras^{G12D}; Trp^{loxP/loxP}; Pdx1-Cre) and KPPC; Col1^{pdxKO} (KPPC; Col1a1^{loxP/loxP}) cells were seeded in to 96-well plates (3×10³ cells per well in 100 μL RPMI with 1% FBS) and then treated with indicated siRNAs for 48 hours. Cell viability/number in each well of 6- or 96-well plates was determined using the Cell Counting Kit-8 (CCK8; Abcam ab228554), examined at OD 450 nm on a microplate reader following the manufacturer's instructions.

Results

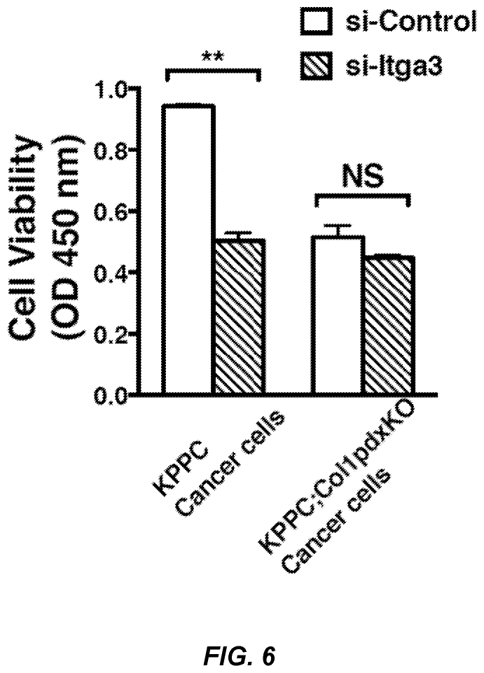

Suppression of a3 integrin by siRNA diminished the proliferation of KPPC cancer cells, but not KPCC; Col1^{pdxKO} (FIG. 6), indicating that interfering with the binding of Col1 homotrimers with α3 integrin on cancer cells impacts their proliferation.

### Example 4—In Vitro Itga3 siRNA Treatment

Methods

Exosomes were produced from human mesenchymal stem cells. 10⁹ number of total exosomes (measured by Nanosight™ analysis) and 1 μg of siRNA (scrambled siRNA-control or siRNA-Itga3) were mixed in 400 μL of electroporation buffer (1.15 mM potassium phosphate pH 7.2, 25 mM potassium chloride, 21% Optiprep). These exosomes were electroporated using a single 4 mm cuvette using a Gene Pulser Xcell Electroporation System (BioRad, 165-2081). The mice were injected with 10⁸ exosomes per injection in 100 μL volume. This dosage represents approximately 0.15 to 0.20 μg of exosome protein load per injection in mice every 48 hours.

Results

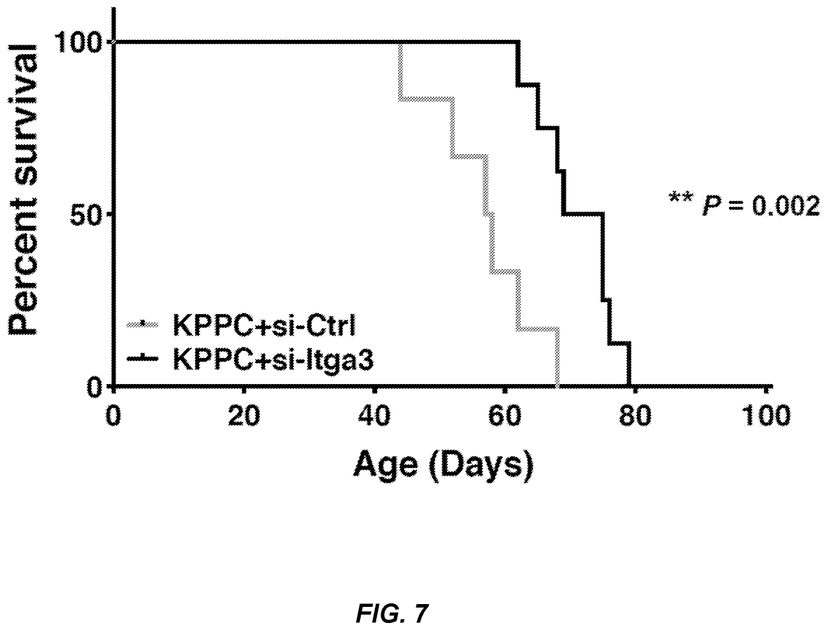

The treatment with exosomes containing siRNA targeting integrin 03 significantly prolonged KPPC mouse survival, as compared with exosomes containing scrambled siRNA control (FIG. 7).

### Example 5—Deletion of Type I Collagen (Col1) in Pancreatic Cancer Cells Increases Accumulation of Immune Cells and Increases Sensitivity to Checkpoint Blockade

Methods

Mice

FSF-Kras^{G12D/+} (5), Pdx1-Flp (5), Trp53^{frt/+} (6), LSL-Kras^{G12D/+} (7), Trp53^{loxP/+} (8), Pdx1-Cre (7), αSMA-Cre (9), and Fsp1-Cre (10, 11) mouse strains were previously documented. Col1a1^{loxP/loxP} mouse strain (with loxP-flanked exons 2-5) was established in the Genetically Engineered Mouse Facility at MD Anderson Cancer Center (MDACC) using the Col1a1^{tm1a(EUCOMM)Wtsi} embryonic stem cells that were obtained from the European Mouse Mutant Cell Repository (EuMMCR).

Rosa26-CAG-loxP-frt-Stop-frt-FireflyLuc-EGFP-loxP-RenillaLuc-tdTomato (referred to as R26^{Dual}) mouse strain contains the novel R26^{Dual} dual-fluorescence reporter allele, which allows the EGFP expression under the control of Pdx1-Flp transgene, or the tdTomato expression under the control of αSMA-Cre and Fsp1-Cre transgenes (12). Characterization of genotyping and disease phenotypes for the FSF-Kras^{G12D/+}; Pdx1-Flp (referred to as KF) or FSF-Kras^{G12D/+}; Trp53^{frt/frt}; Pdx1-Flp (referred to as KPPF) mice was performed as previously described by Saur and colleagues (5). Osteogenesis imperfecta murine (OIM) strain harboring Col1a2 mutation was purchased from Jackson Laboratory (001815; B6C3Fe a/a-Col1a2oim/J). The inventors crossed the KF and KPPF mice with the αSMA-Cre, Pdx1-Cre, Fsp1-Cre, Col1a1^{loxP/loxP}, or R26^{Dual} mouse strains, resulting in the generation of the KF; αSMA-Cre; Col1a1^{loxP/loxP} (referred to as KF; Col1^{smaKO}), KF; Pdx1-Cre; Col1a1^{loxP/loxP} (referred to as KF; Col1^{pdxKO}) KPPF; αSMA-Cre; Col1a1^{loxP/loxP} (referred to as KPPF; Col1^{smaKO}), KPPF; Fsp1-Cre; Col1a1^{loxP/loxP} (referred to as KPPF; Col1^{fspKO}) mice. These mice allow the Col1a1 deletion in either αSMA+ myofibroblasts (MFs) or Fsp1+ cell population in the context of spontaneous PDAC. The KF; Col1^{pdxKO} mouse and the KF; Col1^{smaKO} mouse shared the same control mouse (KF; Cre-negative; Col1a1^{loxP/loxP}), allowing for the direct comparison of disease progression between those three strains (KF control group, KF; Col1^{smaKO} group with Col1 deletion in αSMA-expressing myofibroblasts, and KF; Col1^{pdxKO} group with Col1 deletion in Pdx1-lineage cancer cells). We also crossed the LSL-Kras^{G12D}; Pdx1-Cre (referred to as KC), LSL-Kras^{G12D/+}; Trp53^{R172H/+}; Pdx1-Cre; Col1a1^{loxP/loxP} (referred to as KPC), or LSL-Kras^{G12D}; Trp53^{loxP/loxP}; Pdx1-Cre (referred to as KPPC) mice with the Col1a1^{loxP/loxP} mouse strain, resulting in the generation of the KC; Col1a1^{loxP/loxP} (referred to as KC; Col1^{pdxKO}) KPC; Col1a1^{loxP/loxP} (referred to as KPC; Col1^{pdxKO}), and KPPC; Col1a1^{loxP/loxP} (referred to as KPPC; Col1^{pdxKO} The KF; Col1^{pdxKO}, KC; Col1^{pdxKO}, and KPPC; Col1^{pdxKO} mice allow the Col1a1 deletion in PDAC cells. The aforementioned experimental mice with desired genotypes were monitored and analyzed with no randomization or blinding. Both female and male mice with desired genotype(s) for PDAC were used for experimental mice. Anti-PD1 (BE0273, 29F.1A12, BioXCell) antibodies were intraperitoneally administered at a dose of 100 μg/mouse each, for a total of 3 injections 3 days apart (initiated at 35 days of age). All mice were housed under standard housing conditions at MDACC animal facilities, and all animal procedures were reviewed and approved by the MDACC Institutional Animal Care and Use Committee.

Total mRNA Sequencing

For mRNA sequencing (RNA-seq) analysis of tumor tissues, freshly dissected tumor samples were frozen in RNase-free tubes with liquid nitrogen and preserved at −80° C. Samples were homogenized using bead tubes with ceramic beads on Fisherbrand Bead Mill 24 homogenizer (Fisher Scientific). For RNA-seq of cell lines, KPPC and/or KPPC; Col1^{pdxKO} cancer cells were cultured in 6-well plates (Corning) with vehicle (PBS with 0.5 M Glycerol), homotrimer Col1 (50 μg/mL), or heterotrimer Col1 (50 μg/mL). Cells were harvested after 48 hours of culture. For both tissues and cells, total RNA was extracted using Direct-zol RNA Kit (Zymo Research).

Quality control analysis was conducted using RNA 6000 Nano Kit on Bioanalyzer 2100 (Agilent). Total mRNA sequencing was performed using Illumina TrueSeq stranded mRNAseq Library and High-Output sequencing PE 75×75 nt on NextSeq 500 (Illumina) by MDACC Sequencing and ncRNA Program core facility. Raw sequencing data from the Illumina platform were converted into Fastq files and aligned to the reference genome mm10 using the Spliced Transcripts Alignment to a Reference (STAR) algorithm. HTSeq-count was then utilized to generate the raw counts for each gene. Raw counts were then analyzed by DESeq2 for data processing, normalization, and differential expression analysis according to standard procedures. Functional categorization and pathway reconstitution from the RNA-seq data were conducted using gene set enrichment analysis (GSEA; Broad Institute) and Ingenuity Pathway Analysis (IPA) software (Qiagen). All analyses were implemented in R.

Flow Cytometry

For the characterization of immune infiltration, fresh tumor tissues (from 53-day-old KPPC and KPPC; Col1^{pdxKO} mice, respectively) were weighed, minced with gentleMACS Dissociator, and digested in 2 mL solution containing 1 mg/mL Liberase TL (Roche) and 0.2 mg/mL DNase I in RPMI media at 37° C. for 30 min. The tissue lysates were filtered through a 100 μm mesh before immunostaining. The subsequent single-cells suspension was stained with Fixable Viability Dye eFluor 780 (eBioscience) and appropriate antibodies. Samples were filtered through a 40 μm mesh and examined using a BD LSR Fortessa X20. The percentage positive cells were analyzed by FlowJo 10.1 and gated on CD45 positivity. Unstained, viability stain only, and single-stained beads (eBioscience) were used as compensation controls.

Multispectral Imaging of Multiplex Stained Tissue Sections

The multiplex staining procedures, spectral unmixing and cell segmentation using the Nuance and inForm imaging softwares were described previously (1). Multiplex stained slides were imaged with the Vectra Multispectral Imaging System, using Vectra software version 3.0.3 (Perkin Elmer). Each tissue section was scanned in its entirety using a 4×objective. Up to 80 regions (at 20×) per section were selected for multispectral imaging using the Phenochart software (Perkin Elmer). Each multiplex field was scanned every 10 nm of the emission light spectrum across the range of each emission filter cube. Filter cubes used for multispectral imaging were DAPI (440-600 nm), FITC (520 nm-680 nm), Cy3 (570-690 nm), Texas Red (580-700 nm) and Cy5 (680-720 nm).

Multispectral images from single marker stained slides with the corresponding fluorophores were used to generate a spectral library using the Nuance Image Analysis software (Perkin Elmer). The library contained the emitting spectral peaks of all fluorophores and was used to unmix each multispectral image (spectral unmixing) to its individual 6 components by using the inForm 2.2 image analysis software. Thresholds of detection for the different markers were adjusted across different cohorts in order to ensure consistent capture of positive signal across all controls. All images in each cohort were processed using the same thresholds of staining positivity.

Co-Culture of Mouse Splenic Lymphocytes and PDAC Cancer Cells

KPPC and KPPC; Col1^{pdxKO} cancer cells (2×10⁴ cells/well) were seeded into 96-well plates. Spleen from 4 healthy mice of 2.5-month age (with identical genetic background to KPPC and KPPC; Col1^{pdxKO} mice) was minced, filtered through a 40 μm mesh, washed with ice-cold PBS, and then resuspended in 5 mL of red blood cell lysis solution (sc-296258, Santa Cruz) on ice for 5 min and then washed with PBS. Splenic lymphocytes were counted and seeded into round-bottom 96-well plates (2×10⁵ cells/well) with or without activation for 24 h before being transferred into the 96-well plates containing KPPC or KPPC; Col1^{pdxKO} cancer cells (or only culture medium without cancer cells). After 24 h of co-culture, splenic lymphocytes were harvested, stained using the above method of flow cytometry, and examined for lymphocyte activation. Anti-CD3 (553057, BD Biosciences) and anti-CD28 (553294, BD Biosciences) antibodies (1 μg/mL) were used for the in vitro activation of T cells.

Statistics

Statistical analyses of flow cytometry and immunostaining quantifications were performed with unpaired, two-tailed t test, one-way ANOVA with Tukey's multiple comparison test, or Fisher's exact test using GraphPad Prism (GraphPad Software, San Diego, Calif., USA). χ² analyses were performed comparing metastatic frequency across multiple histological parameters in mice.

Kaplan-Meier plots were drawn for survival analysis and the log rank Mantel-Cox test was used to evaluate statistical differences. Data met the assumptions of each statistical test, where variance was not equal (determined by an F-test) Welch's correction for unequal variances was applied. A P value <0.05 was considered statistically significant. Error bars represented standard error of the mean (S.E.M.) when multiple visual fields were averaged to produce a single value for each animal, which was then averaged again to represent the mean bar for the group in each graph.

Results

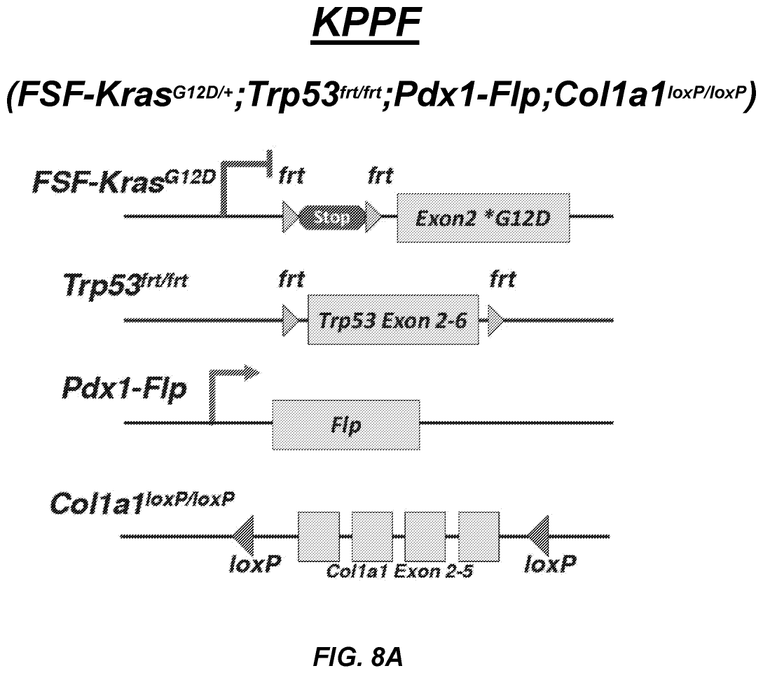

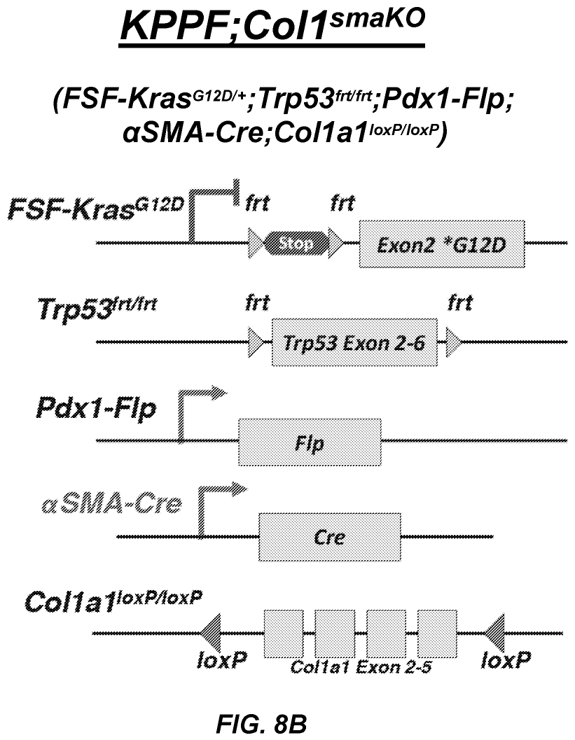

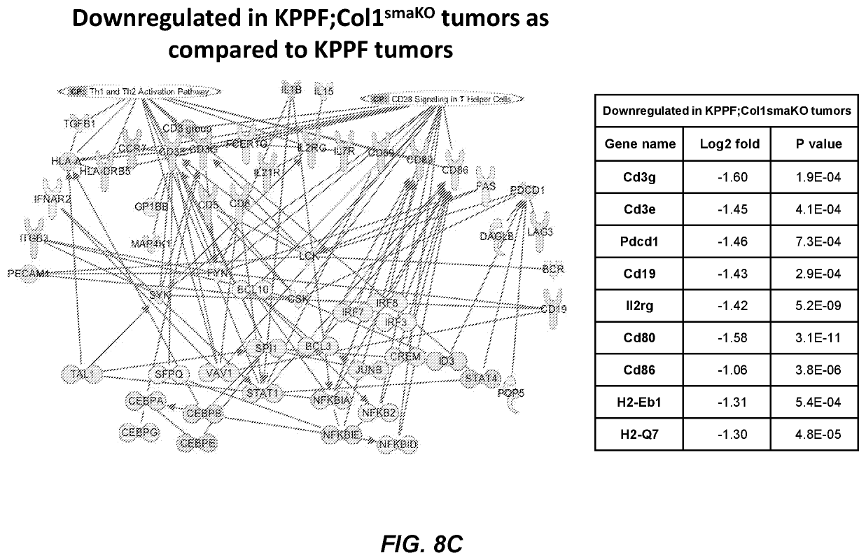

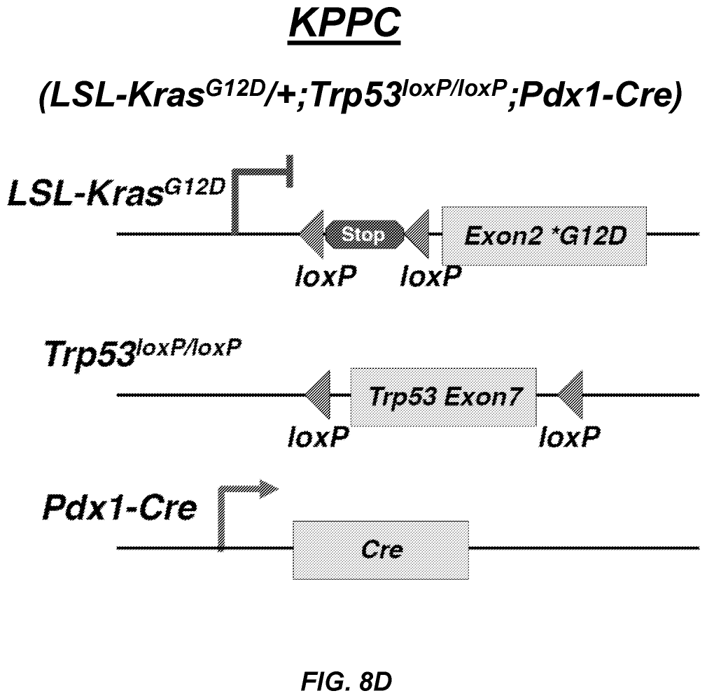

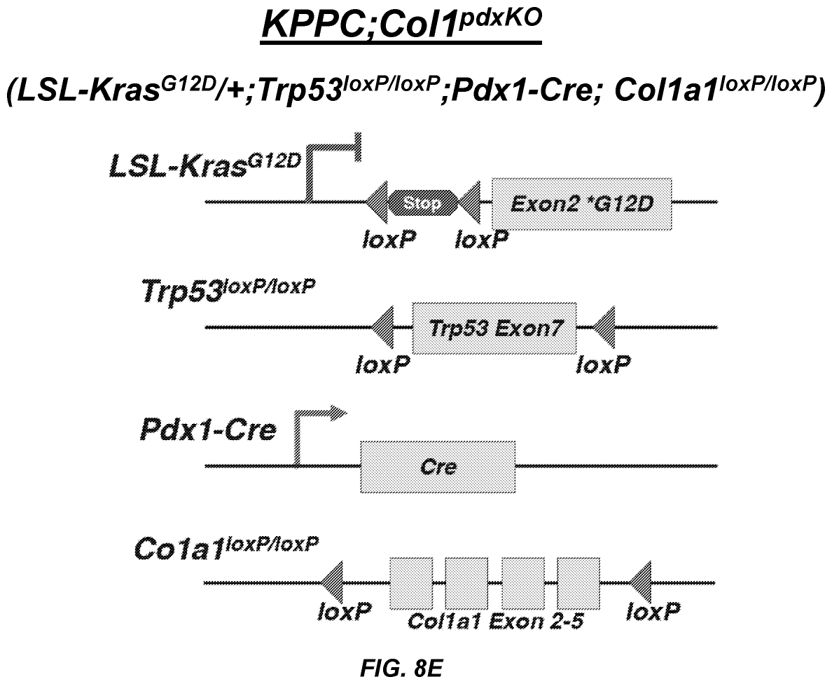

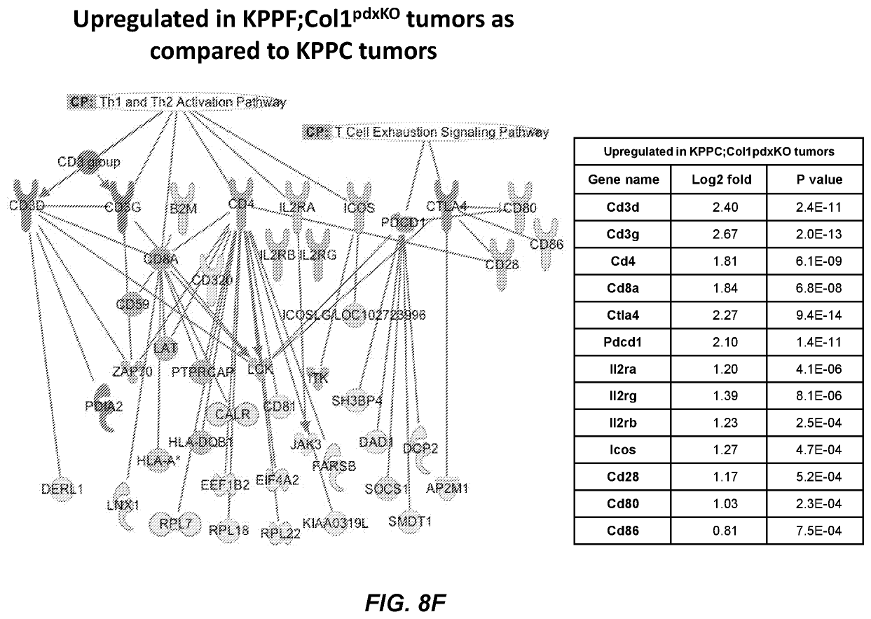

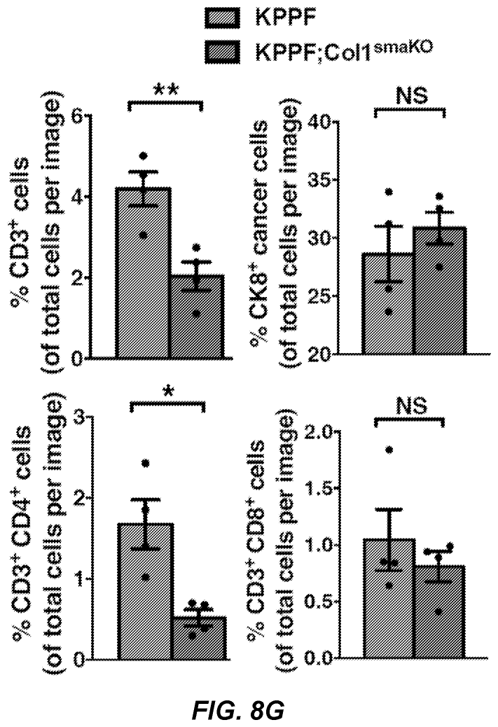

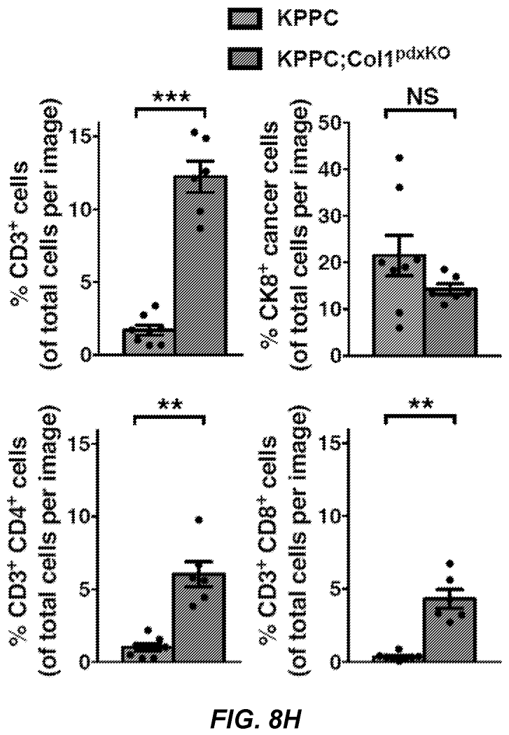

As revealed by gene set enrichment analysis (GSEA), RNA-seq on total RNA from tumor tissues of KPPF; Col1^{smaKO} mice and KPPF control mice revealed significantly downregulated immune response pathways (such as lymphocyte activation/recruitment pathways) in KPPF; Col1^{smaKO} tumors upon the deletion of Col1 in myofibroblasts. Further, ingenuity pathway analysis (IPA) revealed significantly downregulated genes associated with T cell response in in KPPF; Col1^{smaKO} tumors, such as Cd3g, Cd3e, Pdcd1, Il2rg, Cd80, and Cd86 (FIGS. 8A-8C). In contrast, RNA-seq on total RNA from tumor tissues of KPPC; Col1^{pdxKO} mice and KPPC control mice revealed significantly upregulated immune response pathways (such as lymphocyte activation/recruitment pathways) in KPPC; Col1^{pdxKO} tumors upon the deletion of Col1 in cancer cells. IPA revealed significantly upregulated genes associated with T cell response in KPPC; Col1^{pdxKO} tumors, including Cd3d, Cd3g, Cd4, Cd8a, Ctla4, Pdcd1, and Il2ra (FIGS. 8D-8F). Next, tumor sections from KPPC, KPPC; Col1^{pdxKO}, KPPF, and KPPF; Col1^{smaKO} mice were examined by TSA multispectral imaging (1). CD3, CD4 and CD8 T cell infiltration was increased in KPPC; Col1^{pdxKO} tumors but decreased in KPPF; Col1^{smaKO} tumors, when compared to KPPC and KPPF control tumors, respectively (FIGS. 8G and 8H). Taken together, Col1 deletion in myofibroblasts of KPPF; Col1^{smaKO} tumors decreased both stromal Col1 level and T cell infiltration, whereas Col1 deletion in cancer cells of KPPC; Col1^{pdxKO} increased T cell infiltration.

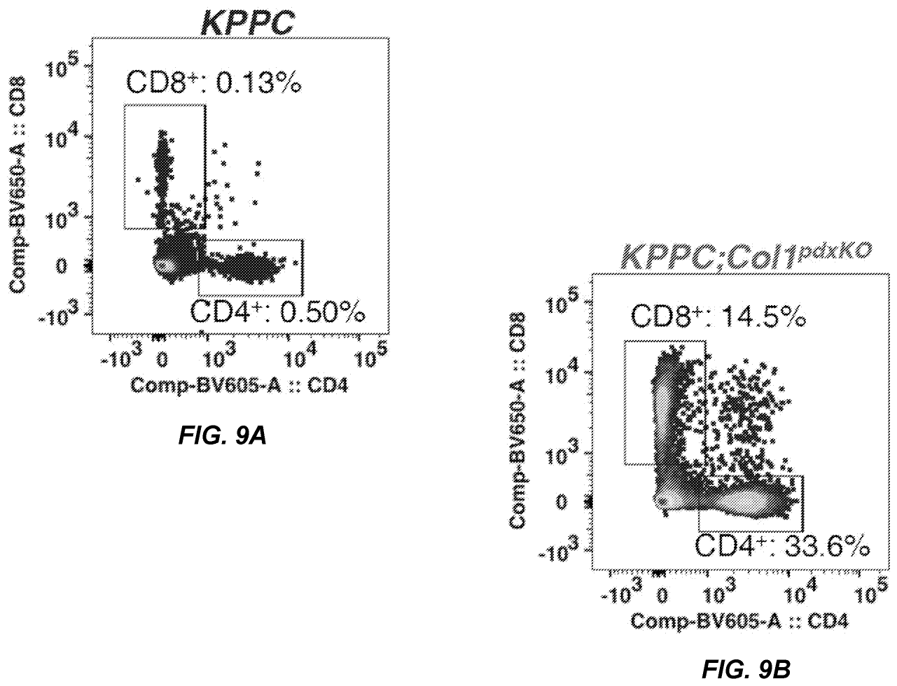

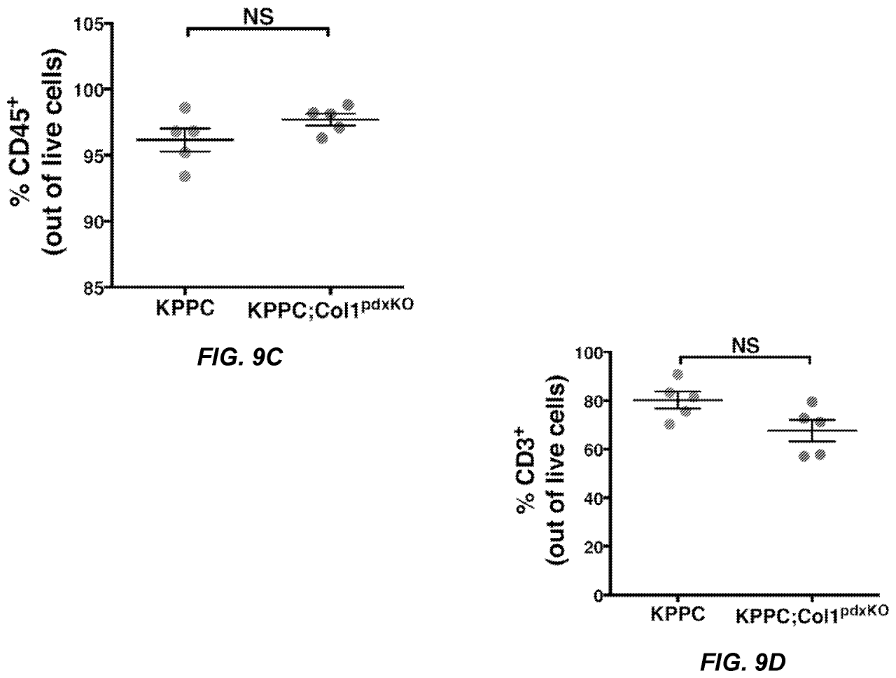

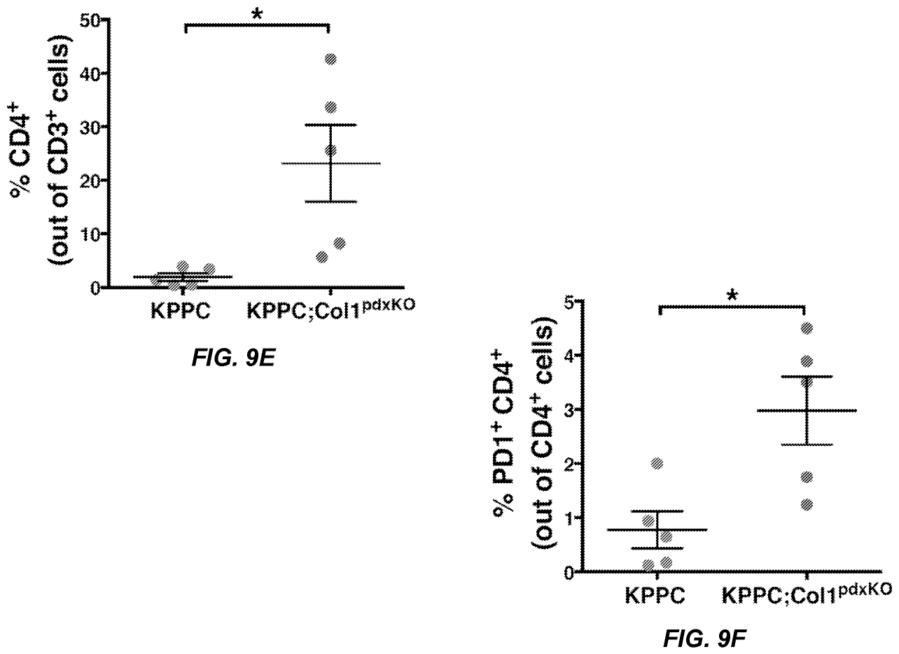

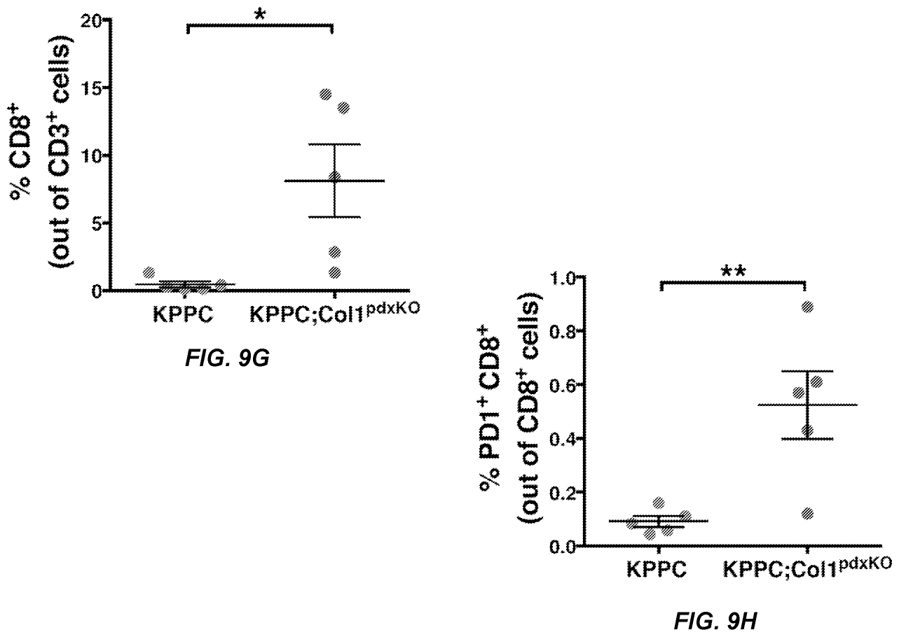

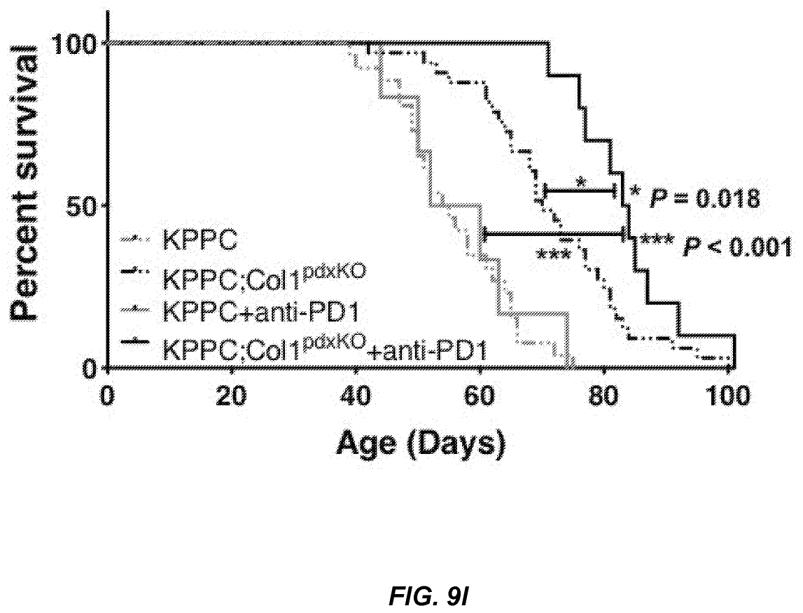

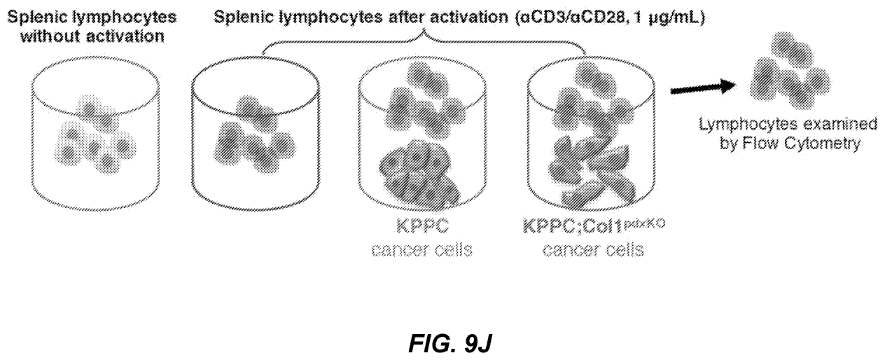

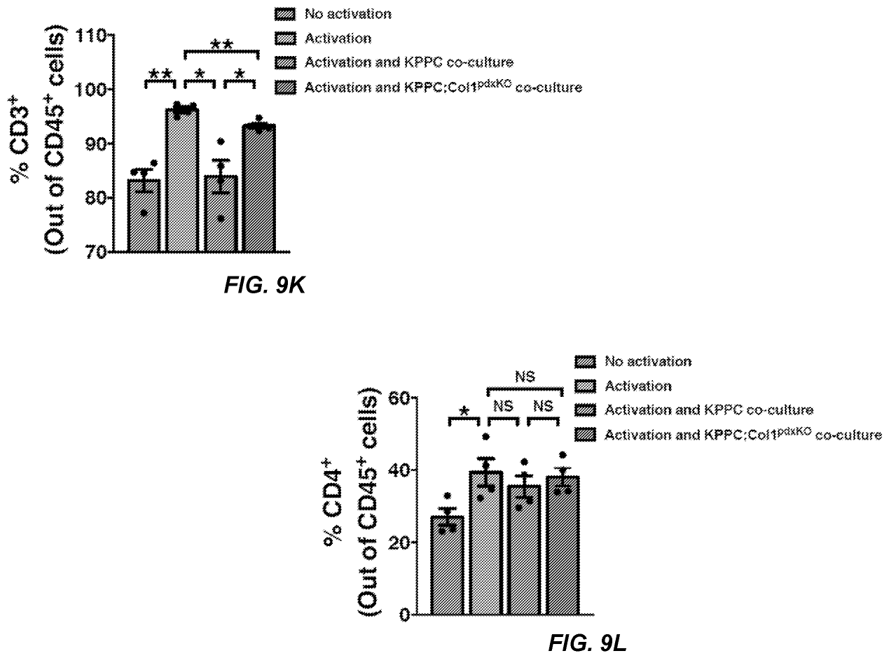

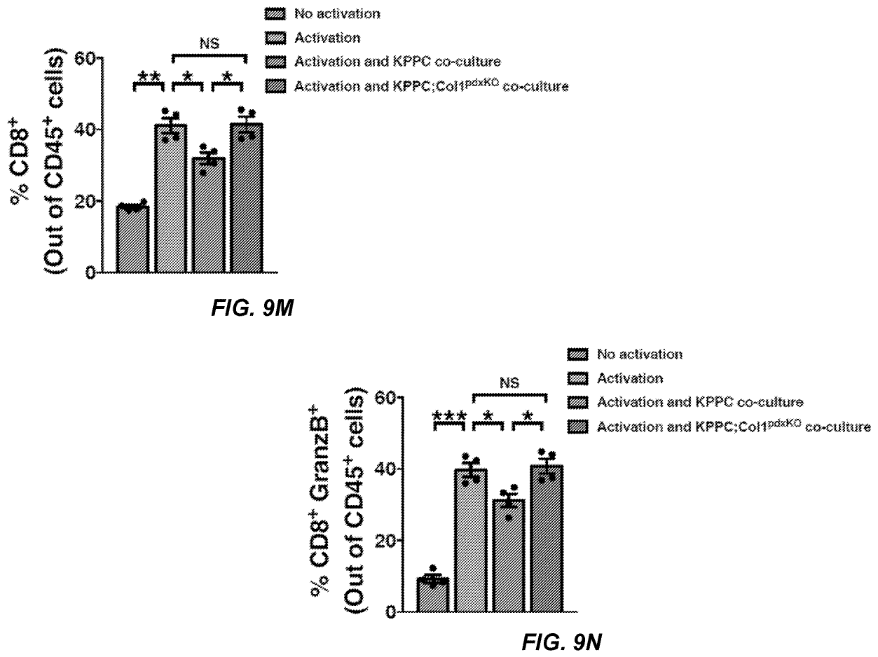

Flow cytometry analyses on fresh tissue demonstrated that KPPC; Col1^{pdxKO} tumors have elevated T cell infiltration and associated T cell activation markers (FIGS. 9A-9H), when compared with KPPC tumors. Specifically, a significant increase in CD4⁺/PD-1⁺ (FIG. 9F) and CD8⁺/PD-1⁺ cells (FIG. 9H) was observed in the KPPC; Col1^{pdxKO} tumors. In order to determine whether such increase in PD-1⁺ T cells of KPPC; Col1^{pdxKO} tumors has any functional significance (2), KPPC mice and KPPC; Col1^{pdxKO} mice with advanced PDAC were treated with anti-PD-1 antibodies. Similar to previous reports (3, 4), KPPC mice were recalcitrant to anti-PD-1 treatment (FIG. 9I). However, KPPC; Col1^{pdxKO} responded positively to anti-PD-1 treatment and exhibited prolonged overall survival (FIG. 9I).

Using in vitro co-culture system of mouse splenic lymphocytes and PDAC cancer cells (a schematic of which is shown in FIG. 9J), it was further demonstrated that KPPC cancer cells significantly suppressed the activation and expansion of co-cultured mouse splenic lymphocytes. In contrast, the KPPC; Col1^{pdxKO} cancer cells, deleted for Col1 homotrimer, lost such immunosuppressive impact on co-cultured mouse splenic lymphocytes (FIG. 9K-9N).

Taken together, these results reveal that oncogenic Col1 homotrimer deletion in cancer cells relieves immunosuppressive PDAC microenvironment, promotes T cell infiltration into the tumors, and enhances efficacy of anti-PD-1 checkpoint blockade therapy.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Apte et al., (2012). Pancreatic stellate cells: a starring role in normal and diseased pancreas. Front Physiol 3, 344.
Armstrong et al., (2004). Type I collagen promotes the malignant phenotype of pancreatic ductal adenocarcinoma. Clinical cancer research: an official journal of the American Association for Cancer Research 10, 7427-7437.
Bachem et al., (2005). Pancreatic carcinoma cells induce fibrosis by stimulating proliferation and matrix synthesis of stellate cells. Gastroenterology 128, 907-921.
Bhowmick et al., (2004). TGF-beta signaling in fibroblasts modulates the oncogenic potential of adjacent epithelia. Science 303, 848-851.
Biffi et al., (2019). IL1-Induced JAK/STAT Signaling Is Antagonized by TGFbeta to Shape CAF Heterogeneity in Pancreatic Ductal Adenocarcinoma. Cancer Discov 9, 282-301.
Bissell & Hines, (2011). Why don't we get more cancer? A proposed role of the microenvironment in restraining cancer progression. Nature medicine 17, 320-329.
Bonnans et al., (2014). Remodelling the extracellular matrix in development and disease. Nature reviews Molecular cell biology 15, 786-801.
Carstens et al., (2017). Spatial computation of intratumoral T cells correlates with survival of patients with pancreatic cancer. Nature communications 8, 15095.
Chen et al., (2018). Dual reporter genetic mouse models of pancreatic cancer identify an epithelial-to-mesenchymal transition-independent metastasis program. EMBO molecular medicine 10.
Chen et al., (2005). Crucial role of p53-dependent cellular senescence in suppression of Pten-deficient tumorigenesis. Nature 436, 725-730.
Chipman et al., (1993). Defective pro alpha 2(I) collagen synthesis in a recessive mutation in mice: a model of human osteogenesis imperfecta. Proceedings of the National Academy of Sciences of the United States of America 90, 1701-1705.
DeClerck et al., (1987). Differential collagen biosynthesis by human neuroblastoma cell variants. Cancer research 47, 6505-6510.
Du et al., (1999). Elevation of alpha2(I) collagen, a suppressor of Ras transformation, is required for stable phenotypic reversion by farnesyltransferase inhibitors. Cancer research 59, 2059-2063.
Egeblad et al., (2010). Dynamic interplay between the collagen scaffold and tumor evolution. Curr Opin Cell Biol 22, 697-706.
Fujita et al., (2009). Tumor-stromal interactions with direct cell contacts enhance proliferation of human pancreatic carcinoma cells. Cancer science 100, 2309-2317.
Haber et al., (1999). Activation of pancreatic stellate cells in human and experimental pancreatic fibrosis. The American journal of pathology 155, 1087-1095.
Han et al., (2010). Molecular mechanism of type I collagen homotrimer resistance to mammalian collagenases. The Journal of biological chemistry 285, 22276-22281.
Han et al., (2008). Segregation of type I collagen homo- and heterotrimers in fibrils. J Mol Biol 383, 122-132.
Hay, (1981). Extracellular matrix. The Journal of cell biology 91, 205s-223s.
Hingorani et al., (2005). Trp53R172H and Kras^{G12D} cooperate to promote chromosomal instability and widely metastatic pancreatic ductal adenocarcinoma in mice. Cancer cell 7, 469-483.
Huang et al., (2015). Ductal pancreatic cancer modeling and drug screening using human pluripotent stem cell- and patient-derived tumor organoids. Nature medicine 21, 1364-1371.
Imamura et al., (1995). Quantitative analysis of collagen and collagen subtypes I, III, and V in human pancreatic cancer, tumor-associated chronic pancreatitis, and alcoholic chronic pancreatitis. Pancreas 11, 357-364.
Jiang et al., (2016). Targeting focal adhesion kinase renders pancreatic cancers responsive to checkpoint immunotherapy. Nature medicine 22, 851-860.
Kalluri, (2016). The biology and function of fibroblasts in cancer. Nature reviews Cancer 16, 582-598.
Kopp et al., (2012). Identification of Sox9-dependent acinar-to-ductal reprogramming as the principal mechanism for initiation of pancreatic ductal adenocarcinoma. Cancer cell 22, 737-750.
Kuznetsova et al., (2001). Osteogenesis imperfecta murine: interaction between type I collagen homotrimers. J Mol Biol 309, 807-815.
Kuznetsova et al., (2004). Structure, stability and interactions of type I collagen with GLY349-CYS substitution in alpha 1(I) chain in a murine Osteogenesis Imperfecta model. Matrix biology: journal of the International Society for Matrix Biology 23, 101-112.
Kuznetsova et al., (2003). Changes in thermal stability and microunfolding pattern of collagen helix resulting from the loss of alpha2(I) chain in osteogenesis imperfecta murine. J Mol Biol 331, 191-200.
Laklai et al., (2016). Genotype tunes pancreatic ductal adenocarcinoma tissue tension to induce matricellular fibrosis and tumor progression. Nature medicine 22, 497-505.
LeBleu et al., (2013). Origin and function of myofibroblasts in kidney fibrosis. Nature medicine 19, 1047-1053.
Lee et al., (2012). Generation of primary tumors with Flp recombinase in FRTflanked p53 mice. Disease models & mechanisms 5, 397-402.
Lee et al., (2014). Stromal response to Hedgehog signaling restrains pancreatic cancer progression. Proceedings of the National Academy of Sciences of the United States of America 111, E3091-3100.
Leitinger, (2011). Transmembrane collagen receptors. Annu Rev Cell Dev Biol 27, 265-290.
Levental et al., (2009). Matrix crosslinking forces tumor progression by enhancing integrin signaling. Cell 139, 891-906.
Lohler et al., (1984). Embryonic lethal mutation in mouse collagen I gene causes rupture of blood vessels and is associated with erythropoietic and mesenchymal cell death. Cell 38, 597-607.
Makareeva et al., (2010). Carcinomas contain a matrix metalloproteinaseresistant isoform of type I collagen exerting selective support to invasion. Cancer research 70, 4366-4374.
Miles et al., (2002). The role of the alpha2 chain in the stabilization of the collagen type I heterotrimer: a study of the type I homotrimer in oim mouse tissues. J Mol Biol 321, 797-805.
Miroshnikova et al., (2016). Tissue mechanics promote IDH1-dependent HIF1 alpha-tenascin C feedback to regulate glioblastoma aggression. Nature cell biology 18, 1336-1345.
Misawa et al., (2011). Hypermethylation of collagen alpha2 (I) gene (COL1A2) is an independent predictor of survival in head and neck cancer. Cancer Biomark 10, 135-144.
Mollenhauer et al., (1987). Distribution of extracellular matrix proteins in pancreatic ductal adenocarcinoma and its influence on tumor cell proliferation in vitro. Pancreas 2, 14-24.
Mouw et al., (2014). Tissue mechanics modulate microRNA-dependent PTEN expression to regulate malignant progression. Nature medicine 20, 360-367.
Mueller & Fusenig, (2004). Friends or foes—bipolar effects of the tumour stroma in cancer. Nature reviews Cancer 4, 839-849.
Neesse et al., (2015). Stromal biology and therapy in pancreatic cancer: a changing paradigm. Gut 64, 1476-1484.
Noda, (1993). Mechanisms of reversion. FASEB J 7, 834-840.
Northcott et al., (2018). Feeling Stress: The Mechanics of Cancer Progression and Aggression. Front Cell Dev Biol 6, 17.
Ohlund et al., (2014). Fibroblast heterogeneity in the cancer wound. The Journal of experimental medicine 211, 1503-1523.
Ohlund et al., (2017). Distinct populations of inflammatory fibroblasts and myofibroblasts in pancreatic cancer. The Journal of experimental medicine 214, 579-596.
Olive et al., (2009). Inhibition of Hedgehog signaling enhances delivery of chemotherapy in a mouse model of pancreatic cancer. Science 324, 1457-1461.
Ozdemir et al., (2014). Depletion of carcinoma-associated fibroblasts and fibrosis induces immunosuppression and accelerates pancreas cancer with reduced survival. Cancer cell 25, 719-734.
Pickup et al., (2013). Stromally derived lysyl oxidase promotes metastasis of transforming growth factor-beta-deficient mouse mammary carcinomas. Cancer research 73, 5336-5346.
Provenzano et al., (2012). Enzymatic targeting of the stroma ablates physical barriers to treatment of pancreatic ductal adenocarcinoma. Cancer cell 21, 418-429.
Rhim et al., (2014). Stromal elements act to restrain, rather than support, pancreatic ductal adenocarcinoma. Cancer cell 25, 735-747.
Ruoslahti, (1991). Integrins. The Journal of clinical investigation 87, 1-5.
Schonhuber et al., (2014). A next-generation dual recombinase system for time- and host-specific targeting of pancreatic cancer. Nature medicine 20, 1340-1347.
Sengupta et al., (2003). DNA hypermethylation near the transcription start site of collagen alpha2(I) gene occurs in both cancer cell lines and primary colorectal cancers. Cancer research 63, 1789-1797.
Seymour et al., (2007). SOX9 is required for maintenance of the pancreatic progenitor cell pool. Proceedings of the National Academy of Sciences of the United States of America 104, 1865-1870.
Strutz et al., (1995). Identification and characterization of a fibroblast marker: FSP1. The Journal of cell biology 130, 393-405.
Sugimoto et al., (2006). Identification of fibroblast heterogeneity in the tumor microenvironment. Cancer biology & therapy 5, 1640-1646.
Takada et al., (2007). The integrins. Genome Biol 8, 215.
Travers et al., (1996). Suppression of tumorigenicity in Ras-transformed fibroblasts by alpha 2(I) collagen. Cell Growth Differ 7, 1353-1360.
von Ahrens et al., (2017). The role of stromal cancer-associated fibroblasts in pancreatic cancer. Journal of hematology & oncology 10, 76.
Xue et al., (2003). The gatekeeper effect of epithelial-mesenchymal transition regulates the frequency of breast cancer metastasis. Cancer research 63, 3386-3394.
Yeh et al., (2012). A tale of two collagen receptors, integrin beta1 and discoidin domain receptor 1, in epithelial cell differentiation. Am J Physiol Cell Physiol 303, C1207-1217.

What is claimed is:

1-10. (canceled)

11. A method of treating a patient in need thereof, the method comprising administering an effective amount of an α3β1 integrin-specific antibody or antibody fragment.

12-13. (canceled)

14. The method of claim 11, wherein the patient has a cancer, a fibroid disease, a tissue injury, keloids, organ fibrosis, Crohn's disease, strictures, colitis, psoriasis, or a connective tissue disorder.

15. The method of claim 14, wherein the connective tissue disorder is a connective tissue disorder that involves collagen.

16. The method of claim 15, wherein the connective tissue disorder that involves collagen is a connective tissue disorder that involves type 1 collagen.

17. The method of claim 15, wherein the patient has a cancer.

18. (canceled)

19. The method of claim 17, wherein the cancer patient has been determined to express an elevated level of α1 homotrimeric type I collagen relative to a control patient.

20. The method of claim 17, wherein the cancer is a pancreatic cancer.

21. The method of claim 20, further defined as a method of inhibiting pancreatic cancer metastasis.

22. The method of claim 20, further defined as a method of inhibiting pancreatic cancer growth.

23. The method of claim 17, further comprising administering at least a second anti-cancer therapy.

24. The method of claim 23, wherein the second anti-cancer therapy is a chemotherapy, immunotherapy, radiotherapy, gene therapy, surgery, hormonal therapy, anti-angiogenic therapy or cytokine therapy.

25. The method of claim 24, wherein the second anti-cancer therapy is an immunotherapy.

26-27. (canceled)

28. The method of claim 17, further comprising administering a FAK and/or PYK2 inhibitor.

29. (canceled)

30. The method of claim 28, further comprising administering VS-4718 (PND-1086).

31-47. (canceled)

48. A chimeric antigen receptor (CAR) polypeptide comprising, from N- to C-terminus, an antigen binding domain; a hinge domain; a transmembrane domain and an intracellular signaling domain, wherein the CAR polypeptide binds to an α3β1 integrin.

49-54. (canceled)

55. A nucleic acid molecule encoding a CAR polypeptide of any one of claims 48-54.

56. (canceled)

57. An isolated immune effector cell comprising the nucleic acid of claim 55.

58-61. (canceled)

62. A pharmaceutical composition comprising the cell of claim 58 and a pharmaceutically acceptable carrier.

63. A method of treating a subject comprising administering the composition of claim 62.

64-92. (canceled)

93. A method of treating a subject for cancer, the method comprising:
(a) administering to the subject an effective amount of an α3β1 integrin-specific antibody or antibody fragment;
(b) administering to the subject an anti-tumor effective amount of chimeric antigen receptor (CAR) T cells that expresses a CAR polypeptide comprising, from N- to C-terminus, an antigen binding domain; a hinge domain; a transmembrane domain and an intracellular signaling domain, wherein the CAR polypeptide binds to an α3β1 integrin; or
(c) administering to the subject an anti-tumor effective amount of an agent that inhibits the expression of α3β1 integrin;
wherein cancer cells from the subject have been determined to have an increased expression of α3 integrin relative to healthy or control cells.

94-123. (canceled)
